SEQUENCE LISTING (40 sequences)

SEQ ID NO: 1 - protein, 132 aa, Bacteriophage Qbeta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 2 - protein, 329 aa, Bacteriophage Qbeta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 3 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 4 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 5 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 6 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 7 - protein, 329 aa, Bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 8 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 9 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 10 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 11 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 12 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 13 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 14 - protein, 132 aa, Bacteriophage Qbeta
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 15 - protein, 132 aa, Bacteriophage Qbeta
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 16 - protein, 132 aa, Bacteriophage Qbeta
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 17 - protein, 132 aa, Bacteriophage Qbeta
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 18 - protein, 132 aa, Bacteriophage Qbeta
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 19 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 20 - DNA, 348 bp, Dengue virus
catatggataaactgaccctgaaaggcatgagctatgtgatgtgcaccggcagcttcaaactggaaaaagaagtggcggaaacccagcatggcaccgtgctggtgcaggtgaaatatgaaggcaccgatgcgccgtgcaaaattccgtttagcacccaggatgaaaaaggcgtgacccagaacggccgtctgattaccgcgaatccgattgtgaccgataaagaaaaaccggtgaacattgaaaccgaaccgccgtttggcgaaagctatattgtggtgggtgcgggcgaaaaagcgctgaaactgagctggtttaaaaaaggcagcagcattggcaaaatgctcgag

SEQ ID NO: 21 - protein, 113 aa, Dengue virus
MDKLTLKGMSYVMCTGSFKLEKEVAETQHGTVLVQVKYEGTDAPCKIPFSTQDEKGVTQNGRLITANPIVTDKEKPVNIETEPPFGESYIVVGAGEKALKLSWFKKGSSIGKM

SEQ ID NO: 22 - protein, 124 aa, Dengue virus
MDKLTLKGMSYVMCTGSFKLEKEVAETQHGTVLVQVKYEGTDAPCKIPFSTQDEKGVTQNGRLITANPIVTDKEKPVNIETEPPFGESYIVVGAGEKALKLSWFKKGSSIGKMLEHHHHHHGGC

SEQ ID NO: 23 - DNA, 348 bp, Dengue virus
catatggacaaactacagctcaaaggaatgtcatactctatgtgcacaggaaagtttaaagttgtgaaggaaatagcagaaacacaacatggaacaatagttatcagagtacaatatgaaggggacggttctccatgtaagatcccttttgagataatggatttggaaaaaagacatgttttaggtcgcctgattacagtcaacccaatcgtaacagaaaaagatagcccagtcaacatagaagcagaacctccattcggagacagctacatcatcataggagtagagccgggacaattgaagctcaactggtttaagaaaggaagttctatcggccaaatgctcgag

SEQ ID NO: 24 - protein, 113 aa, Dengue virus
MDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQM

SEQ ID NO: 25 - protein, 124 aa, Dengue virus
MDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMLEHHHHHHGGC

SEQ ID NO: 26 - DNA, 348 bp, Dengue virus
catatggataaactggaactgaaaggcatgagctatgccatgtgcctgaacacctttgtgctgaaaaaagaagtgagcgaaacccagcatggcaccattctgatcaaagtggaatataaaggcgaagatgcgccgtgcaaaattccgtttagcaccgaagatggccagggcaaagcgcataacggccgtctgattaccgcgaatccggtggtgaccaaaaaagaagaaccggttaacattgaagcggaaccgccgtttggcgaaagcaacattgtgattggcattggcgataaagcgctgaaaatcaactggtacaaaaaaggcagcagcattggcaaaatgctcgag

SEQ ID NO: 27 - protein, 113 aa, Dengue virus
MDKLELKGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYKKGSSIGKM

SEQ ID NO: 28 - protein, 124 aa, Dengue virus
MDKLELKGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYKKGSSIGKMLEHHHHHHGGC

SEQ ID NO: 29 - DNA, 348 bp, Dengue virus
catatggaaaaactgcgcattaaaggcatgagctataccatgtgcagcggcaaatttagcatcgataaagaaatggcggaaacccagcatggcaccaccgtggtgaaagtgaaatatgaaggcgcgggtgcgccgtgcaaagtgccgattgaaattcgtgatgtgaacaaagaaaaagtggtgggccgtattattagcagcaccccgtttgcggaaaacaccaacagcgtgaccaacattgaactggaaccgccgtttggcgatagctatattgtgattggcgtgggcgatagcgcgctgaccctgcattggtttcgtaaaggcagcagcattggcaaaatgctcgag

SEQ ID NO: 30 - protein, 113 aa, Dengue virus
MEKLRIKGMSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGKM

SEQ ID NO: 31 - protein, 124 aa, Dengue virus
MEKLRIKGMSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGKMLEHHHHHHGGC

SEQ ID NO: 32 - protein, 113 aa, Dengue virus
MEKLRIKGMSYTMCPGKFSIDKEMAETQHGTTVVKVKYEGAGAPCKVPIEIRDVNKEKVVGRIISSTPLAENTNSATNIELEPPFGDSYIVIGVGNSALTLHWFRKGSSIGKM

SEQ ID NO: 33 - protein, 118 aa, Dengue virus
MDKLTLKGMSYVMCTGSFKLEKEVAETQHGTVLVQVKYEGTDAPCKIPFSTQDEKGVTQNGRLITANPIVTDKEKPVNIETEPPFGESYIVVGAGEKALKLSWFKKGSSIGKMGGSCG

SEQ ID NO: 34 - protein, 118 aa, Dengue virus
MDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMGGSCG

SEQ ID NO: 35 - protein, 118 aa, Dengue virus
MDKLELKGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYKKGSSIGKMGGSCG

SEQ ID NO: 36 - protein, 118 aa, Dengue virus
MEKLRIKGMSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGKMGGSCG

SEQ ID NO: 37 - protein, 104 aa, Dengue virus
MSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSGSCG

SEQ ID NO: 38 - protein, 108 aa, Dengue virus
MSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGKGSCG

SEQ ID NO: 39 - protein, 104 aa, Dengue virus
MSYTMCPGKFSIDKEMAETQHGTTVVKVKYEGAGAPCKVPIEIRDVNKEKVVGRIISSTPLAENTNSATNIELEPPFGDSYIVIGVGNSALTLHWFRKGSGSCG

SEQ ID NO: 40 - protein, 108 aa, Dengue virus
MSYTMCPGKFSIDKEMAETQHGTTVVKVKYEGAGAPCKVPIEIRDVNKEKVVGRIISSTPLAENTNSATNIELEPPFGDSYIVIGVGNSALTLHWFRKGSSIGKGSCG

(19) United States
(12) Patent Application Publication
(10) Pub. No.: US 2011/0262472 A1
(43) Pub. Date: Oct. 27, 2011

(54) VACCINE COMPOSITIONS FOR THE TREATMENT OF DENGUE FEVER AND USES THEREOF

(75) Inventors: Martin Bachmann, Seuzach (CH); Adrian Huber, Zurich (CH); Lidia Ivanova, Schlieren (CH); Martin Bachmann, Seuzach (CH)

(73) Assignee: CYTOS BIOTECHNOLOGY AG

(21) Appl. No.: 12/989,186

(22) PCT Filed: Apr. 22, 2009
(86) PCT No.: PCT/EP2009/054856

§ 371 Date: Jun. 6, 2011

(30) Foreign Application Priority Data
Apr. 22, 2008 (EP) 08154974.3

(51) Int. Cl. A61K 39/12; A61P 31/14

(52) U.S. Cl. 424/186.1; 424/218.1

(57) ABSTRACT

The invention provides compositions, vaccine compositions and pharmaceutical compositions for the treatment, amelioration and/or prevention of dengue fever. The compositions, vaccine compositions and pharmaceutical compositions of the invention comprise a virus-like particle of an RNA bacteriophage and at least one antigen, wherein said at least one antigen is a dengue antigen. When administered to an animal, preferably to a human, said compositions, vaccine compositions and pharmaceutical compositions induce efficient immune responses, in particular antibody responses, wherein typically and preferably said antibody responses are directed against dengue virus, preferably against dengue virus of any one of serotypes 1 to 4. Thus, the invention further provides methods of treating, ameliorating and/or preventing dengue virus infection by way of active immunization against domain III of the dengue virus envelope protein E, or against antigenic fragments thereof.

### FIELD OF THE INVENTION

The present invention is in the fields of medicine, public health, immunology, molecular biology and virology. The invention provides compositions, vaccine compositions and pharmaceutical compositions for the treatment, amelioration and/or prevention of dengue fever. The compositions, vaccine compositions and pharmaceutical compositions of the invention comprise a virus-like particle of an RNA bacteriophage and at least one antigen, wherein said at least one antigen is a dengue antigen. When administered to an animal, preferably to a human, said compositions, vaccine compositions and pharmaceutical compositions induce efficient immune responses, in particular antibody responses, wherein typically and preferably said antibody responses are directed against dengue virus, preferably against dengue virus of any one of serotypes 1 to 4. Thus, the invention further provides methods of treating, ameliorating and/or preventing dengue virus infection by way of active immunization against domain III of the dengue virus envelope protein E, or against antigenic fragments thereof.

### BACKGROUND OF THE INVENTION

Dengue infection is a major public health problem. There are approximately 50-100 million cases of dengue fever, 500,000 cases of dengue hemorrhagic fever (DHF) and dengue shock syndrome (DSS), and more than 20,000 death each year. Most infections are asymptomatic or cause a nonfatal debilitating illness, but especially children often experience the more fatal forms DHF and DSS. Mortality may be reduced to less than 1% if the lost body fluids are replaced by transfusion. The primary dengue vector is a mosquito (Aedes aegypti) that has is found throughout the tropics. Since there is no specific treatment available, the only method to control dengue is to combat the mosquito.

Dengue viruses belong to the family of Flaviviridae. There are four closely related dengue serotypes (serotypes 1 to 4). Flaviviruses have a diameter of 50 nm and contain a single stranded RNA genome that is surrounded by a capsid, a lipid bilayer, and an icosahedral shell on the particle surface made of two glycoproteins. A single open reading frame codes for a long polyprotein that is cleaved into three structural (nucleocapsid protein C, membrane precursor protein prM, and envelope protein E) and seven non-structural proteins (NS1, NS2A, NS2B, NS3, NS4A, NS4B, NS5). prM and NS1 have been targets for neutralising antibodies (Kaufman et al. 1989 μm. J. Trop. Med. Hyg. 41(5):576-580; Henchal et al. 1988 J. Gen. Virol. 69:2101-2107; Wu et al. 2003 Vaccine 21:3919-3929), but many studies have also focused on the envelope protein E. In the mature virion, E is a membrane associated homodimeric glycoprotein that mediates viral attachment and interaction with cellular receptors on target cells. The crystal structure of the soluble ectodomain of E was solved (Modis et al. 2003 Proc. Natl. Acad. Sci. USA. 100:6986-6991) and shown to comprise three domains: domain I is located centrally, domain II contains the fusion peptide and a dimerisation region, and domain III confers the receptor binding activity.

Dengue envelope domain III has been suggested to contain neutralising epitopes (Roehrig et al. 1990 Virology 177:668-675; Megret al. 1992 Virology 187:480-491; Trirawatanapong et al. 1992 Gene 116:139-150). Since there is no specific therapy, vaccination remains the most promising route for controlling dengue infection (Ray and Shi, 2006 Recent Patents Anti-Infect Drug Disc. 1:45-55).

Sugrue et al. 1997 (J. Gen. Virol. 78:1861-1866) have expressed the dengue virus structural proteins in Pichia pastoris (C-prM-E). The budding virus-like particles were checked by SDS-PAGE and transmission electron microscopy, where they observed spherical structures, whose morphology resembled dengue virions. The virus-like particles were immunogenic in animals and were able to induce neutralizing antibodies.

Virus-like particles (VLPs) of parvovirus B19 (non-enveloped, single-stranded DNA virus) carrying DEN-2-specific epitopes were used as antigen carriers (Amexis and Young, 2006 J. Infect. Dis. 194:790-794). Two peptides of domain III and the full-length domain III of the envelope glycoprotein (residues 352-368 and 386-397) were displayed as recombinant fusions on B19 VLPs.

In another study, it was shown that the dengue envelope protein (residues 1-395) could be expressed in yeast as a fusion protein to the hepatitis B surface antigen (Bisht et al. 2002 J. Biotechnol. 99:97-110). Serum from immunised mice was shown to bind to the envelope protein and to dengue infected cells.

In yet another study, recombinant proteins containing the B domain of dengue virus serotypes 1-4 fused to the maltose binding protein (MBP) of Escherichia coli were evaluated individually and as a tetravalent vaccine candidate in mice (Simmons et al. 2001 Am. J. Trop. Med. Hyg. 65:159-161).

### SUMMARY OF THE INVENTION

We have found that the inventive compositions and vaccine compositions, respectively, comprising or consisting of domain III of the dengue virus envelope protein E or an antigenic fragment thereof are not only capable of inducing immune responses against dengue virus envelope protein E, and hereby in particular antibody responses, but are, furthermore, capable of neutralizing dengue virus in Plaque Reduction Neutralization test (PRNT50), as described in Russell et al. 1967 (Journal of Immunology 99, 291-296).

Thus, one aspect of the invention is a composition comprising: (a) a virus-like particle with at least one first attachment site, wherein said virus-like particle is a virus-like particle of an RNA bacteriophage; and (b) at least one antigen with at least one second attachment site, wherein said at least one antigen is a dengue antigen, wherein said dengue antigen comprises at least position 9 to 99 of domain III of the dengue virus envelope protein E; and wherein (a) and (b) are linked through said at least one first and said at least one second attachment site. In a very preferred embodiment, said at least one antigen with said at least one second attachment site comprises or preferably consists of any one of SEQ ID NOs 22, 25, 28 and 31, and wherein preferably said domain III of the dengue virus envelope protein E is selected from the group consisting of: (i) domain III of the dengue virus envelope protein E of a dengue virus of serotype-1; (ii) domain III of the dengue virus envelope protein E of a dengue virus of serotype-2; (iii) domain III of the dengue virus envelope protein E of a dengue virus of serotype-3; and (iv) domain III of the dengue virus envelope protein E of a dengue virus of serotype-4.

In a preferred embodiment of the present invention, said dengue antigen comprises, or preferably consists of, at least position 9 to 99 of domain III of the dengue virus envelope protein E of a dengue virus of serotype-1. In another preferred embodiment of the present invention, said dengue antigen comprises, or preferably consists of, at least position 9 to 99 of domain III of the dengue virus envelope protein E of a dengue virus of serotype-2. In a further preferred embodiment of the present invention, said dengue antigen comprises, or preferably consists of, at least position 9 to 99 of domain III of the dengue virus envelope protein E of a dengue virus of serotype-3. In another preferred embodiment of the present invention, said dengue antigen comprises, or preferably consists of, at least position 9 to 99 of domain III of the dengue virus envelope protein E of a dengue virus of serotype-4.

In a preferred embodiment of the present invention, said dengue antigen comprises, or preferably consists of, position 9 to 109 or position 9 to 112 of domain III of the dengue virus envelope protein E. In another preferred embodiment of the present invention, said dengue antigen comprises or preferably consists of position 9 to 99 of any one of SEQ ID NOs 21, 24, 27, 30 and 32. In a further preferred embodiment of the present invention, said dengue antigen comprises or preferably consists of position 9 to 109 or position 9 to 112 of any one of the SEQ ID NOs 21, 24, 27, 30 and 32.

In another aspect of the present invention, the invention provides for a composition comprises, or preferably consists of, (i) a first composition, (ii) a second composition, (iii) a third composition, and (iv) a fourth composition, wherein said first composition (i) comprises a virus-like particle of an RNA bacteriophage with at least one first attachment site and at least one antigen with at least one second attachment site, wherein said at least one antigen is a dengue antigen comprising, or preferably consisting of, at least position 9 to 99 of domain III of the dengue virus envelope protein E of a dengue virus of serotype-1; and wherein said second composition (ii) comprises a virus-like particle of an RNA bacteriophage with at least one first attachment site and at least one antigen with at least one second attachment site, wherein said at least one antigen is a dengue antigen comprising, or preferably consisting of, at least position 9 to 99 of domain III of the dengue virus envelope protein E of a dengue virus of serotype-2; and wherein said third composition (iii) comprises a virus-like particle of an RNA bacteriophage with at least one first attachment site and at least one antigen with at least one second attachment site, wherein said at least one antigen is a dengue antigen comprising, or preferably consisting of, at least position 9 to 99 of domain III of the dengue virus envelope protein E of a dengue virus of serotype-3; and wherein said fourth composition (iv) comprises a virus-like particle of an RNA bacteriophage with at least one first attachment site and at least one antigen with at least one second attachment site, wherein said at least one antigen is a dengue antigen comprising, or preferably consisting of, at least position 9 to 99 of domain III of the dengue virus envelope protein E of a dengue virus of serotype-4; and wherein said virus-like particles of an RNA bacteriophage and said at least one antigen of each of said first composition, said second composition, said third composition and said fourth composition are linked through said at least one first and said at least one second attachment site.

A further aspect of the invention is a vaccine composition comprising, or alternatively consisting of a composition of the invention.

A further aspect of the invention is a pharmaceutical composition comprising: (a) a composition or a vaccine composition of the invention; and (b) a pharmaceutically acceptable carrier.

A further aspect of the invention is a method of treating, ameliorating, or preventing dengue fever, dengue hemorrhagic fever, and/or dengue shock syndrome, said method comprising administering an immunologically effective amount of the composition, of the vaccine composition, and/or of the pharmaceutical composition of the invention to an animal, preferably to a human.

A further aspect of the invention is the use of the composition, of the vaccine composition and/or of the pharmaceutical composition of the invention for the manufacture of a medicament for treatment, amelioration and/or prevention of dengue fever, dengue hemorrhagic fever, and/or dengue shock syndrome, preferably in an animal, most preferably in a human.

A further aspect of the invention is the composition, the vaccine composition, and/or the pharmaceutical composition of the invention for the treatment, amelioration and/or prevention of dengue fever, dengue hemorrhagic fever, and/or dengue shock syndrome in an animal, preferably in a human.

### DETAILED DESCRIPTION OF THE INVENTION

Unless defined otherwise, all technical and scientific terms used herein have the same meanings as commonly understood by one of ordinary skill in the art to which this invention belongs.

Adjuvant: The term “adjuvant” as used herein refers to non-specific stimulators of the immune response or substances that allow generation of a depot in the host which when combined with the vaccine composition and pharmaceutical composition, respectively, of the present invention may provide for an even more enhanced immune response. Preferred adjuvants are complete and incomplete Freund's adjuvant, aluminum containing adjuvant, preferably aluminium hydroxide (Alum), and modified muramyldipeptide. Further preferred adjuvants are mineral gels such as aluminum hydroxide, surface active substances such as lysolecithin, pluronic polyols, polyanions, peptides, oil emulsions, keyhole limpet hemocyanins, dinitrophenol, and human adjuvants such as BCG (bacille Calmette Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminum salts (Alum), MF-59, OM-174, OM-197, OM-294, and Virosomal adjuvant technology. The adjuvants can also comprise a mixture of these substances. VLP have been generally described as an adjuvant. However, the term “adjuvant”, as used within the context of this application, refers to an adjuvant not being the VLP used for the inventive compositions, rather it relates to an additional, distinct component.

Antigen: As used herein, the term “antigen” refers to a molecule capable of being bound by an antibody or a T-cell receptor (TCR) if presented by MHC molecules. The term “antigen”, as used herein, also refers to T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens. The term “antigen” as used herein preferably refers to a dengue antigen, wherein said dengue antigen preferably is a surface antigen of dengue virus, most preferably domain III of the dengue virus envelope protein E or an antigenic fragment thereof. If not indicated otherwise, the term “antigen” as used herein does not refer to the virus-like particle contained in the inventive compositions.

Dengue antigen: as used herein, the term “dengue antigen” refers to domain III of the dengue virus envelope protein E or to antigenic fragments thereof, wherein preferably said dengue antigen comprises or preferably consists of domain III of the dengue virus envelope protein E of a dengue virus of serotype-1, serotype-2, serotype-3, or serotype-4, or of an antigenic fragment thereof. In a preferred embodiment, said dengue antigen comprises or preferably consists at least of position 9 to 99, position 9 to 109 or position 9 to 112 of domain III of the dengue virus envelope protein E, wherein preferably said dengue virus envelope protein E is of a dengue virus of serotype-1, serotype-2, serotype-3, or serotype-4. In another preferred embodiment of the invention, said dengue antigen comprises, or preferably consists of, an amino acid sequence starting with the first amino acid at any one of the position 1, 2, 3, 4, 5, 6, 7, 8, 9, 10 or 11 and ending with the last amino acid at any one of the position 97, 98, 99, 100, 101, 102, 103, 104, 105, 106, 107, 108, 109, 110, 111, 112 or 113 of domain III of the dengue virus envelope protein E, wherein preferably said dengue virus envelope protein E is of a dengue virus of serotype-1, serotype-2, serotype-3, or serotype-4. In a very preferred embodiment, said dengue antigen comprises or preferably consists of position 9 to 99, position 9 to 109 or position 9 to 112 of any one of SEQ ID NOs 21, 24, 27, 30 and 32. In a still more preferred embodiment, said dengue antigen comprises or preferably consists of any one of SEQ ID NOs 21, 24, 27, 30 and 32. The term “dengue antigen” also refers to an antigen comprising or preferably consisting of a polypeptide having at least 90%, preferably at least 95%, more preferably at least 98%, and most preferably at least 99% sequence identity with position 9 to 99, position 9 to 109 or position 9 to 112 of domain III of a dengue virus envelope protein E, wherein preferably said dengue virus envelope protein E is of a dengue virus of serotype-1, serotype-2, serotype-3, or serotype-4. The term “dengue antigen” further refers to an antigen comprising or preferably consisting of a polypeptide having at least 90%, preferably at least 95%, more preferably at least 98%, and most preferably at least 99% sequence identity with position 9 to 99, position 9 to 109 or position 9 to 112 of any one of SEQ ID NOs 21, 24, 27, 30 and 32. Furthermore, term “dengue antigen” refers to an antigen comprising or preferably consisting of a polypeptide having at least 90%, preferably at least 95%, more preferably at least 98%, and most preferably at least 99% sequence identity with any one of SEQ ID NOs 21, 24, 27, 30 and 32.

Epitope: The term epitope refers to continuous or discontinuous portions of an antigen, preferably a polypeptide, wherein said portions can be specifically bound by an antibody or by a T-cell receptor within the context of an MHC molecule. With respect to antibodies, specific binding excludes non-specific binding but does not necessarily exclude cross-reactivity. An epitope typically comprise 5-10 amino acids in a spatial conformation which is unique to the antigenic site.

Associated: The terms “associated” or “association” as used herein refer to all possible ways, preferably chemical interactions, by which two molecules are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds.

Attachment Site, First: As used herein, the phrase “first attachment site” refers to an element which is naturally occurring with the VLP or which is artificially added to the VLP, and to which the second attachment site may be linked. The first attachment site preferably is a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the first attachment site is the amino group of an amino acid, preferably of lysine. The first attachment site is located, typically on the surface, and preferably on the outer surface of the VLP. Multiple first attachment sites are present on the surface, preferably on the outer surface of virus-like particle, typically in a repetitive configuration. In a preferred embodiment the first attachment site is associated with the VLP, through at least one covalent bond, preferably through at least one peptide bond. In a further preferred embodiment the first attachment site is naturally occurring with the VLP. Alternatively, in a preferred embodiment the first attachment site is artificially added to the VLP.

Attachment Site, Second: As used herein, the phrase “second attachment site” refers to an element which is naturally occurring with or which is artificially added to the antigen and to which the first attachment site may be linked. The second attachment site of the antigen preferably is a protein, a polypeptide, a peptide, an amino acid, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the second attachment site is a sulfhydryl group, preferably a sulfhydryl group of an amino acid, most preferably a sulfhydryl group of a cysteine. The term “antigen with at least one second attachment site” refers, therefore, to a construct comprising the antigen and at least one second attachment site. However, in particular for a second attachment site, which is not naturally occurring within the antigen, such a construct typically and preferably further comprises a “linker”. In another preferred embodiment the second attachment site is associated with the antigen through at least one covalent bond, preferably through at least one peptide bond. In a further embodiment, the second attachment site is naturally occurring within the antigen. In another further preferred embodiment, the second attachment site is artificially added to the antigen through a linker, wherein said linker comprises or alternatively consists of a cysteine. Preferably the linker is fused to the antigen by way of a peptide bond.

Coat protein: The term “coat protein” refers to a viral protein, preferably a subunit of a natural capsid of a virus, preferably of a RNA bacteriophage, which is capable of being incorporated into a virus capsid or a VLP.

Linker: A “linker”, as used herein, either associates the second attachment site with the antigen or already comprises, essentially consists of, or consists of the second attachment site. Preferably, a “linker”, as used herein, already comprises the second attachment site, typically and preferably—but not necessarily—as one amino acid residue, preferably as a cysteine residue. A “linker” as used herein is also termed “amino acid linker”, in particular when a linker according to the invention contains at least one amino acid residue. Thus, the terms “linker” and “amino acid linker” are interchangeably used herein. However, this does not imply that such a linker consists exclusively of amino acid residues, even if a linker consisting of amino acid residues is a preferred embodiment of the present invention. The amino acid residues of the linker are, preferably, composed of naturally occurring amino acids or unnatural amino acids known in the art, all-L or all-D or mixtures thereof. Further preferred embodiments of a linker in accordance with this invention are molecules comprising a sulfhydryl group or a cysteine residue and such molecules are, therefore, also encompassed within this invention. Further linkers useful for the present invention are molecules comprising a C1-C6 alkyl-, a cycloalkyl such as a cyclopentyl or cyclohexyl, a cycloalkenyl, aryl or heteroaryl moiety. Moreover, linkers comprising preferably a C1-C6 alkyl-, cycloalkyl-(C5, C6), aryl- or heteroaryl-moiety and additional amino acid(s) can also be used as linkers for the present invention and shall be encompassed within the scope of the invention. Association of the linker with the antigen is preferably by way of at least one covalent bond, more preferably by way of at least one peptide bond. In the context of linkage by genetic fusion, a linker may be absent or preferably is an amino acid linker, more preferably an amino acid linker consisting exclusively of amino acid residues. Very preferred linkers for genetic fusion are flexible amino acid linkers. In the context of linkage by genetic fusion preferred linkers consist of 1 to 20, more preferably of 2 to 15, still more preferably of 2 to 10, still more preferably of 2 to 5, and most preferably of 3 amino acids. Very preferred linkers for genetic fusion comprise or preferably consist of the amino acid sequence GSG.

Ordered and repetitive antigen array: As used herein, the term “ordered and repetitive antigen array” generally refers to a repeating pattern of antigen or, characterized by a typically and preferably high order of uniformity in spacial arrangement of the antigens with respect to virus-like particle, respectively. In one embodiment of the invention, the repeating pattern may be a geometric pattern. Certain embodiments of the invention, such as antigens coupled to the VLP of RNA bacteriophages, are typical and preferred examples of suitable ordered and repetitive antigen arrays which, moreover, possess strictly repetitive paracrystalline orders of antigens, preferably with spacing of 1 to 30 nanometers, preferably 2 to 15 nanometers, even more preferably 2 to 10 nanometers, even again more preferably 2 to 8 nanometers, and further more preferably 1.6 to 7 nanometers.

Packaged: The term “packaged” as used herein refers to the state of a polyanionic macromolecule or immunostimulatory substances in relation to the VLP. The term “packaged” as used herein includes binding that may be covalent, e.g., by chemically coupling, or non-covalent, e.g., ionic interactions, hydrophobic interactions, hydrogen bonds, etc. The term also includes the enclosement, or partial enclosement, of a polyanionic macromolecule. Thus, the polyanionic macromolecule or immunostimulatory substances can be enclosed by the VLP without the existence of an actual binding, in particular of a covalent binding. In preferred embodiments, the at least one polyanionic macromolecule or immunostimulatory substances is packaged inside the VLP, most preferably in a non-covalent manner. In case said immunostimulatory substances is a nucleic acid, preferably a DNA, the term packaged implies that said nucleic acid is not accessible to nucleases hydrolysis, preferably not accessible to DNAse hydrolysis (e.g. DNaseI or Benzonase), wherein preferably said accessibility is assayed as described in Examples 11-17 of WO2003/024481A2.

Polypeptide: The term “polypeptide” as used herein refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. Post-translational modifications of the polypeptide, for example, glycosylations, acetylations, phosphorylations, and the like are also encompassed.

Recombinant VLP: The term “recombinant VLP”, as used herein, refers to a VLP that is obtained by a process which comprises at least one step of recombinant DNA technology. The term “VLP recombinantly produced”, as used herein, refers to a VLP that is obtained by a process which comprises at least one step of recombinant DNA technology. Thus, the terms “recombinant VLP” and “VLP recombinantly produced” are interchangeably used herein and should have the identical meaning.

Virus-like particle (VLP), as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. Preferably a virus-like particle in accordance with the invention is non-replicative and/or non-infectious since it lacks all or part of the viral genome or genome function. In one embodiment, a virus-like particle is a virus particle, in which the viral genome has been physically or chemically inactivated. Typically and more preferably a virus-like particle lacks all or part of the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, preferably RNA bacteriophage. The terms “viral capsid” or “capsid”, refer to a macromolecular assembly composed of viral protein subunits. Typically, there are 60, 120, 180, 240, 300, 360 and more than 360 viral protein subunits. Typically and preferably, the interactions of these subunits lead to the formation of viral capsid or viral-capsid like structure with an inherent repetitive organization, wherein said structure is, typically, spherical or tubular. For example, the capsids of RNA bacteriophages have a spherical form of icosahedral symmetry. The term “capsid-like structure” as used herein, refers to a macromolecular assembly composed of viral protein subunits resembling the capsid morphology in the above defined sense but deviating from the typical symmetrical assembly while maintaining a sufficient degree of order and repetitiveness. One feature of a virus-like particle is its highly ordered and repetitive arrangement of its subunits.

Virus-like particle of an RNA bacteriophage: As used herein, the term “virus-like particle of an RNA bacteriophage” refers to a virus-like particle comprising, or preferably consisting essentially of or consisting of coat proteins, mutants or fragments thereof, of an RNA bacteriophage. In addition, virus-like particle of an RNA bacteriophage resembling the structure of an RNA bacteriophage, being non replicative and/or non-infectious, and lacking at least the gene or genes encoding for the replication machinery of the RNA bacteriophage, and typically also lacking the gene or genes encoding the protein or proteins responsible for viral attachment to or entry into the host. This definition should, however, also encompass virus-like particles of RNA bacteriophages, in which the aforementioned gene or genes are still present but inactive, and, therefore, also leading to non-replicative and/or non-infectious virus-like particles of an RNA bacteriophage. Preferred VLPs derived from RNA bacteriophages exhibit icosahedral symmetry and consist of 180 subunits (monomers). Preferred methods to render a virus-like particle of an RNA bacteriophage non replicative and/or non-infectious is by physical, chemical inactivation, such as UV irradiation, formaldehyde treatment, typically and preferably by genetic manipulation.

One, a, or an: when the terms “one”, “a”, or “an” are used in this disclosure, they mean “at least one” or “one or more” unless otherwise indicated.

Sequence Identity (amino acid sequences): The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, polypeptides or a fragment thereof disclosed in this invention.

The compositions described herein are capable of inducing and/or enhancing an immune responses against dengue virus in an animal or in human. It has been found that immunization of mice with a composition of the invention resulted in PRNT50 titers that were between 67 and 5475 (cf. Example 8). In one aspect, the invention provides a composition comprising: (a) a virus-like particle with at least one first attachment site, wherein said virus-like particle is a virus-like particle of an RNA bacteriophage; and (b) at least one antigen with at least one second attachment site, wherein said at least one antigen is a dengue antigen, wherein said dengue antigen comprises or preferably consists at least of position 9 to 99, position 9 to 109 or position 9 to 112 of domain III of the dengue virus envelope protein E, wherein preferably said dengue virus envelope protein E is of a dengue virus of serotype-1, serotype-2, serotype-3, or serotype-4; and wherein (a) and (b) are linked through said at least one first and said at least one second attachment site. Preferably, said antigen is linked to the virus-like particle, so as to form an ordered and repetitive antigen-VLP array.

In a preferred embodiment, said dengue antigen comprises or preferably consists of position 9 to 99, position 9 to 109 or position 9 to 112 of any one of SEQ ID NOs 21 (domain III of envelope protein E of strain Reunion 305/04; Swissprot: A0S5S5; serotype-1), 24 (domain III of envelope protein E of strain Thailand/NGS-C/1944; Swissprot: P14340; serotype-2), 27 (domain III of envelope protein E of strain Singapore/8120/1995; Swissprot: Q5UB51; serotype-3), 30 (domain III of envelope protein E of strain MY01-23314; Swissprot: Q8B0G5; serotype-4) and 32 (domain III of envelope protein E of strain Indonesia 1976; NCBI: U18429; serotype-4).

In a further preferred embodiment said dengue antigen comprises or preferably consists of domain III of the dengue virus envelope protein E of a dengue virus, wherein preferably said dengue virus is of serotype-1, serotype-2, serotype-3, or serotype-4.

In a further preferred embodiment said dengue antigen comprises or preferably consists of any one of SEQ ID NOs 21, 24, 27, 30 and 32.

In a very preferred embodiment said dengue antigen consists of SEQ ID NO:24.

In preferred embodiments of the invention, at least 20, preferably at least 30, more preferably at least 60, again more preferably at least 120, and still more preferably at least 180 dengue antigens are linked to the virus-like particle.

Virus-like particles can be produced and purified from virus-infected cell cultures. For the purpose of vaccination, the resulting virus-like particles should be preferably non-replicative or non-infectious, more preferably non-replicative and non-infectious. UV irradiation, chemical treatment, such as with formaldehyde or chloroform, are the general methods known to skilled person in the art to inactivate a virus.

In one preferred embodiment, the VLP is a recombinant VLP. Almost all commonly known viruses, and in particular RNA bacteriophages, have been sequenced and are readily available to the public. The gene encoding the coat protein can be easily identified by a skilled artisan. The preparation of VLPs by recombinantly expressing the coat protein in a host is within the common knowledge of the artisan.

The term “fragment of a recombinant protein” or the term “fragment of a coat protein”, as used herein, is defined as a polypeptide, which is at least 70%, preferably at least 80%, more preferably at least 90%, even more preferably at least 95% of the length of the wild-type recombinant protein, or coat protein, respectively, and which preferably retains the capability of forming a VLP. Preferably, the fragment is obtained by at least one internal deletion, at least one truncation or at least one combination thereof. Further preferably, the fragment is obtained by at most 5, 4, 3 or 2 internal deletions, by at most 2 truncations or by exactly one combination thereof.

The term “fragment of a recombinant protein” or “fragment of a coat protein” shall further refer to a polypeptide, which has at least 80%, preferably at least 90%, more preferably at least 95% amino acid sequence identity with the “fragment of a recombinant protein” or “fragment of a coat protein”, respectively, as defined above and which is preferably capable of assembling into a virus-like particle.

The term “mutant coat protein” refers to a polypeptide having an amino acid sequence derived from the wild type recombinant protein, or coat protein, respectively, wherein the amino acid sequence is at least 80%, preferably at least 85%, 90%, 95%, 97%, or 99% identical to the wild type sequence, and wherein preferably said amino acid sequence retains the ability to assemble into a VLP.

It is a specific advantage of coat proteins of RNA bacteriophages that they can readily be expressed in bacterial expression systems, in particular in E. coli. Thus, in one preferred embodiment of the invention, the virus-like particle comprises, consists essentially of, or alternatively consists of, recombinant coat proteins, mutants or fragments thereof, of an RNA bacteriophage. Preferably, the RNA bacteriophage is selected from the group consisting of: (a) bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (k) bacteriophage f2; (1) bacteriophage PP7; and (m) bacteriophage AP205.

In one preferred embodiment of the invention, the virus-like particle comprises coat proteins, mutants or fragments thereof, of RNA bacteriophages, wherein said coat proteins comprise or preferably consists of an amino acid sequence selected from the group consisting of: (a) SEQ ID NO:1, referring to Qβ CP; (b) a mixture of SEQ ID NO:1 and SEQ ID NO:2 (Qβ A1 protein); (c) SEQ ID NO:3 (R17 coat protein); (d) SEQ ID NO:4 (fr coat protein); (e) SEQ ID NO:5 (GA coat protein); (f) SEQ ID NO:6 (SP coat protein); (g) a mixture of SEQ ID NO:6 and SEQ ID NO:7; (h) SEQ ID NO:8 (MS2 coat protein); (i) SEQ ID NO:9 (M11 coat protein); (j) SEQ ID NO:10 (MX1 coat protein); (k) SEQ ID NO:11 (NL95 coat protein); (1) SEQ ID NO:12 (f2 coat protein); (m) SEQ ID NO:13 (PP7 coat protein); and (n) SEQ ID NO:19 (AP205 coat protein).

In one preferred embodiment of the invention, the VLP is a mosaic VLP comprising or alternatively consisting of more than one amino acid sequence, preferably two amino acid sequences, of coat proteins, mutants or fragments thereof, of an RNA bacteriophage.

In one very preferred embodiment, the VLP comprises or alternatively consists of two different coat proteins of an RNA bacteriophage, wherein said two different coat proteins comprise or preferably consist of the amino acid sequence of CP Qβ (SEQ ID NO:1) and CP Qβ A1 (SEQ ID NO:2); or of the amino acid sequence of CP SP (SEQ ID NO:6) and CP SP A1 (SEQ ID NO:7).

In preferred embodiments of the present invention, the virus-like particle comprises, or alternatively consists essentially of, or alternatively consists of recombinant coat proteins, mutants or fragments thereof, of an RNA bacteriophage, wherein preferably said RNA bacteriophage is selected from bacteriophage Qβ, bacteriophage fr, bacteriophage AP205, and bacteriophage GA.

In one preferred embodiment of the invention, the VLP is a VLP of RNA bacteriophage Qβ. The capsid or virus-like particle of Qβ shows an icosahedral phage-like capsid structure with a diameter of 25 nm and T=3 quasi symmetry. The capsid contains 180 copies of the coat protein, which are linked in covalent pentamers and hexamers by disulfide bridges (Golmohammadi, R. et al., Structure 4:543-5554 (1996)), leading to a remarkable stability of the Qβ capsid. Capsids or VLPs made from recombinant Qβ coat protein may contain, however, subunits not linked via disulfide bonds to other subunits within the capsid, or incompletely linked. The capsid or VLP of Qβ shows unusual resistance to organic solvents and denaturing agents. Surprisingly, we have observed that DMSO and acetonitrile concentrations as high as 30%, and guanidinium concentrations as high as 1 M do not affect the stability of the capsid. The high stability of the capsid or VLP of Qβ is an advantageous feature, in particular, for its use in immunization and vaccination of mammals and humans in accordance of the present invention.

Further preferred virus-like particles of RNA bacteriophages, in particular of bacteriophage Qβ and bacteriophage fr, are disclosed in WO 02/056905, the disclosure of which is herewith incorporated by reference in its entirety. In particular Example 18 of WO 02/056905 contains a detailed description of the preparation of VLP particles of bacteriophage Qβ.

In another preferred embodiment, the VLP is a VLP of bacteriophage AP205. Assembly-competent mutant forms of AP205 VLPs, including AP205 coat protein with the substitution of proline at amino acid 5 to threonine, may also be used in the practice of the invention and leads to other preferred embodiments of the invention. WO 2004/007538 describes, in particular in Example 1 and Example 2, how to obtain VLP comprising AP205 coat proteins, and hereby in particular the expression and the purification thereof. WO 2004/007538 is incorporated herein by way of reference. AP205 VLPs are highly immunogenic, and can be linked with the antigen to typically and preferably generate constructs displaying the antigen oriented in a repetitive manner.

In one preferred embodiment, the VLP comprises, essentially consists of, or alternatively consists of a mutant coat protein of an RNA bacteriophage, wherein the mutant coat protein has been modified by removal of at least one lysine residue by way of substitution and/or by way of deletion. In another preferred embodiment, the VLP comprises, essentially consists of, or alternatively consists of a mutant coat protein of an RNA bacteriophage, wherein said mutant coat protein has been modified by addition of at least one lysine residue by way of substitution and/or by way of insertion. The deletion, substitution or addition of at least one lysine residue allows varying the degree of coupling, i.e. the amount of antigen per subunit of the VLP, preferably of the VLP of an RNA bacteriophage, in particular, to match and tailor the requirements of the vaccine.

In one preferred embodiment, the compositions and vaccine compositions of the invention have an antigen density being from 0.5 to 4.0. The term “antigen density”, as used herein, refers to the average number of antigen molecules which is linked per subunit, preferably per coat protein, of the VLP, and hereby preferably of the VLP of an RNA bacteriophage. Thus, this value is calculated as an average over all the subunits of the VLP, preferably of the VLP of the RNA bacteriophage, in the composition or vaccine compositions of the invention.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. Preferably, the at least one first attachment site is a lysine residue, pointing to or being on the exterior of the VLP.

Qβ mutants, of which exposed lysine residues are replaced by arginines can be used for the present invention. Thus, in another preferred embodiment of the present invention, the virus-like particle comprises, consists essentially of, or alternatively consists of mutant Qβ coat proteins. Preferably these mutant coat proteins comprise or alternatively consist of an amino acid sequence selected from the group of (a) Qβ-240 (SEQ ID NO:14, Lys13-Arg of SEQ ID NO:1); (b) Qβ-243 (SEQ ID NO:15, Asn10-Lys of SEQ ID NO:1); (c) Qβ-250 (SEQ ID NO:16, Lys2-Arg of SEQ ID NO:1); (d) Qβ-251 (SEQ ID NO:17, Lys16-Arg of SEQ ID NO:1); and (e) Qβ-259 (SEQ ID NO:18, Lys2-Arg, Lys16-Arg of SEQ ID NO:1). The construction, expression and purification of the above indicated Qβ mutant coat proteins, mutant Qβ coat protein VLPs and capsids, respectively, are described in WO 02/056905. In particular is hereby referred to Example 18 of above mentioned application.

In another preferred embodiment of the present invention, the virus-like particle comprises, or alternatively consists essentially of, or alternatively consists of mutant coat protein of Qβ, or of fragments thereof, and the corresponding A1 protein. In a further preferred embodiment, the virus-like particle comprises, or alternatively consists essentially of, or alternatively consists of mutant coat protein with amino acid sequence SEQ ID NO:14, 15, 16, 17, or 18 and the corresponding A1 protein.

Further RNA bacteriophage coat proteins have also been shown to self-assemble upon expression in a bacterial host (Kastelein, R A. et al., Gene 23:245-254 (1983), Kozlovskaya, T M. et al., Dokl. Akad. Nauk SSSR 287:452-455 (1986), Adhin, M R. et al., Virology 170:238-242 (1989), Priano, C. et al., J. Mol. Biol. 249:283-297 (1995)). In particular the biological and biochemical properties of GA (Ni, CZ., et al., Protein Sci. 5:2485-2493 (1996), Tars, K et al., J. Mol. Biol. 271:759-773 (1997)) and of fr (Pushko P. et al., Prot. Eng. 6:883-891 (1993), Liljas, L et al. J. Mol. Biol. 244:279-290, (1994)) have been disclosed. The crystal structure of several RNA bacteriophages has been determined (Golmohammadi, R. et al., Structure 4:543-554 (1996)). Using such information, surface exposed residues can be identified and, thus, RNA bacteriophage coat proteins can be modified such that one or more reactive amino acid residues can be inserted by way of insertion or substitution. Another advantage of the VLPs derived from RNA bacteriophages is their high expression yield in bacteria that allows production of large quantities of material at affordable cost.

In one preferred embodiment, the composition of the invention comprises at least one antigen, preferably one to four, more preferably one to three, still more preferably one to two and most preferably exactly one antigen, wherein said antigen is dengue antigen.

Further disclosed is a method of producing the compositions of the invention comprising (a) providing a virus-like particle with at least one first attachment site, wherein said virus-like particle is a virus-like particle of an RNA bacteriophage; (b) providing at least one antigen with at least one second attachment site, wherein said at least one antigen is a dengue antigen, and wherein preferably said dengue antigen comprises at least position 9 to 99, position 9 to 109 or position 9 to 112 of domain III of the dengue virus envelope protein E; and (c) combining said virus-like particle and said at least one antigen to produce said composition, wherein said at least one antigen and said virus-like particle are linked through the first and the second attachment sites. In a preferred embodiment, the provision of the at least one antigen with the at least one second attachment site is by way of expression, preferably by way of expression in a bacterial system, preferably in E. coli. Usually a purification tag, such as His tag, Myc tag, Fc tag or HA tag is added to facilitate the purification process. In another approach the dengue antigens are chemically synthesized.

In one preferred embodiment of the invention, the VLP with at least one first attachment site is linked to the antigen with at least one second attachment site via at least one peptide bond. A gene encoding an antigen, preferably a dengue antigen, is in-frame ligated, either internally or preferably to the N- or the C-terminus to the gene encoding the coat protein of the virus-like particle. Fusion may also be effected by inserting sequences of the antigen into a mutant coat protein where part of the coat protein sequence has been deleted, that are further referred to as truncation mutants. Truncation mutants may have N- or C-terminal, or internal deletions of part of the sequence of the coat protein. The fusion protein shall preferably retain the ability of assembly into a VLP upon expression which can be examined by electromicroscopy.

Flanking amino acid residues may be added to increase the distance between the coat protein and foreign epitope. Glycine and serine residues are particularly favored amino acids to be used in the flanking sequences. Such a flanking sequence confers additional flexibility, which may diminish the potential destabilizing effect of fusing a foreign sequence into the sequence of a VLP subunit and diminish the interference with the assembly by the presence of the foreign epitope.

In other embodiments, the at least one antigen, preferably the dengue antigen, can be fused to a number of other viral coat protein, as way of examples, to the C-terminus of a truncated form of the A1 protein of Qβ (Kozlovska, T. M., et al., Intervirology 39:9-15 (1996)), or being inserted between position 72 and 73 of the CP extension. As another example, the dengue antigen can be inserted between amino acid 2 and 3 of the fr CP, leading to a dengue antigen-fr CP fusion protein (Pushko P. et al., Prot. Eng. 6:883-891 (1993)). Furthermore, dengue antigen can be fused to the N-terminal protuberant β-hairpin of the coat protein of RNA bacteriophage MS-2 (WO 92/13081).

U.S. Pat. No. 5,698,424 describes a modified coat protein of bacteriophage MS-2 capable of forming a capsid, wherein the coat protein is modified by an insertion of a cysteine residue into the N-terminal hairpin region, and by replacement of each of the cysteine residues located external to the N-terminal hairpin region by a non-cysteine amino acid residue. The inserted cysteine may then be linked directly to a desired molecular species to be presented such as an epitope or an antigenic protein.

We note, however, that the presence of an exposed free cysteine residue in the capsid may lead to oligomerization of capsids by way of disulfide bridge formation. Moreover, attachment between capsids and antigenic proteins by way of disulfide bonds are labile, in particular, to sulfhydryl-moiety containing molecules, and are, furthermore, less stable in serum than, for example, thioether attachments (Martin F J. and Papahadjopoulos D. (1982) Irreversible Coupling of Immunoglobulin Fragments to Preformed Vesicles. J. Biol. Chem. 257: 286-288).

Therefore, in a further very preferred embodiment, the association or linkage of the VLP and the at least one antigen, i.e. the dengue antigen, does not comprise a disulfide bond. Further preferred hereby, the at least one second attachment comprise, or preferably is, a sulfhydryl group. Moreover, in again a very preferred embodiment of the present invention, the association or linkage of the VLP and the at least one antigen does not comprise a sulphur-sulphur bond. Further preferred hereby, the at least one second attachment comprise, or preferably is, a sulfhydryl group. In a further very preferred embodiment, said at least one first attachment site is not or does not comprise a sulfhydryl group. In again a further very preferred embodiment, said at least one first attachment site is not or does not comprise a sulfhydryl group of a cysteine.

In a further preferred embodiment said at least one first attachment comprises an amino group and said second attachment comprises a sulfhydryl group.

In a further preferred embodiment, said first attachment is an amino group and said second attachment site is a sulfhydryl group. In a still further preferred embodiment, said first attachment is an amino group of a lysine, and said second attachment site is a sulfhydryl group of a cysteine.

In a further preferred embodiment only one of said second attachment sites associates with said first attachment site through at least one non-peptide covalent bond leading to a single and uniform type of binding of said antigen to said virus-like particle, wherein said only one second attachment site that associates with said first attachment site is a sulfhydryl group, and wherein said antigen and said virus-like particle interact through said association to form an ordered and repetitive antigen array.

In a further preferred embodiment said virus-like particle comprises, essentially consists of, or alternatively consists of, recombinant coat proteins, mutants or fragments thereof, of an RNA bacteriophage, wherein said at least one antigen is fused to the N- or the C-terminus of said recombinant coat proteins, mutants or fragments thereof.

In a further preferred embodiment said virus-like particle comprises, essentially consists of, or alternatively consists of, recombinant coat proteins, mutants or fragments thereof, of an RNA bacteriophage, wherein preferably said RNA bacteriophage is selected from the group consisting of: (a) bacteriophage AP205; (b) bacteriophage fr; and (c) bacteriophage GA; and wherein said at least one antigen is fused to the N- or the C-terminus, preferably to the C-terminus, of said recombinant coat proteins, mutants or fragments thereof, and wherein further preferably said at least one antigen is a dengue antigen, wherein said dengue antigen comprises at least position 9 to 99, position 9 to 109 or position 9 to 112 of domain III of the dengue virus envelope protein E, and wherein still more preferably said dengue antigen comprises or preferably consists of position 9 to 99, position 9 to 109 or position 9 to 112 of any one of SEQ ID NOs 21, 24, 27, 30 and 32.

In a further preferred embodiment said virus-like particle comprises, essentially consists of, or alternatively consists of, recombinant coat proteins, mutants or fragments thereof, of an RNA bacteriophage, wherein preferably said RNA bacteriophage is selected from the group consisting of: (a) bacteriophage AP205; (b) bacteriophage fr; and (c) bacteriophage GA; and wherein said at least one antigen is fused to the N- or the C-terminus, preferably to the C-terminus, of said recombinant coat proteins, mutants or fragments thereof, and wherein further preferably said at least one antigen is a dengue antigen, wherein said dengue antigen comprises or preferably consists of any one of SEQ ID NOs 21, 24, 27, 30 and 32.

In a further preferred embodiment said at least one antigen is fused to either the N- or the C-terminus, preferably the C-terminus, of a coat protein, mutants or fragments thereof, of RNA bacteriophage AP205, wherein preferably said at least one antigen comprises or preferably consists of position 9 to 99, position 9 to 109 or position 9 to 112 of any one of SEQ ID NOs 21, 24, 27, 30 and 32, and wherein further preferably said dengue antigen comprises or preferably consists of any one of SEQ ID NOs 21, 24, 27, 30 and 32.

The term “capable of inducing a balanced immune response” is defined as an ability of a tetravalent composition (containing four compositions wherein each composition also referred to as “first”, “second”, “third” and “fourth” composition relates to a virus-like particle of RNA bacteriophage linked to a domain III of the dengue virus envelope protein E of serotype-1, serotype-2, serotype-3 and serotype-4 respectively) to neutralize all of the four dengue serotypes in a mammal to a similar extent as exhibited by a monovalent composition (containing a virus-like particle of RNA bacteriophage linked to a dengue antigen of any one of the respective serotype-1, serotype-2, serotype-3 or serotype-4 of domain III of the dengue virus envelope protein E) for the respective serotype. The neutralizing ability is measured using a PRNT50 assay which is performed according to Russell et al. 1967 (Journal of Immunology 99, 291-296).

The term “similar extent” used in the context of “capable of inducing a balanced immune response” refers to the values of the neutralization titers (PRNT50) as described for instance in Example 7 and 9 of the present invention, wherein the comparison of the titer values of monovalent composition vs tetravalent composition of the invention does not differ more than 1 log₁₀, more preferably 0.5 log₁₀, most preferably 0.25 log₁₀.

VLPs comprising fusion proteins of coat protein of bacteriophage AP205 with an antigen are generally disclosed in WO2006/032674A1 which is incorporated herein by reference. In one further preferred embodiment, the fusion protein further comprises a linker, wherein said linker is fused to the coat protein, fragments or mutants thereof, of AP205 and the antigen. In a further preferred embodiment said antigen is fused to the C-terminus of said coat protein, fragments or mutants thereof, of AP205 via said linker.

In one preferred embodiment of the present invention, the composition comprises or alternatively consists essentially of a virus-like particle with at least one first attachment site linked to at least one antigen, i.e. a dengue antigen according to the invention, with at least one second attachment site via at least one covalent bond, wherein preferably the covalent bond is a non-peptide bond. In a preferred embodiment of the present invention, the first attachment site comprises, or preferably is, an amino group, preferably the amino group of a lysine residue. In another preferred embodiment of the present invention, the second attachment site comprises, or preferably is, a sulfhydryl group, preferably a sulfhydryl group of a cysteine.

In a very preferred embodiment of the invention, the at least one first attachment site is an amino group, preferably an amino group of a lysine residue and the at least one second attachment site is a sulfhydryl group, preferably a sulfhydryl group of a cysteine.

In one preferred embodiment of the invention, the antigen is linked to the VLP by way of chemical cross-linking, typically and preferably by using a heterobifunctional cross-linker. In preferred embodiments, the hetero-bifunctional cross-linker contains a functional group which can react with the preferred first attachment sites, preferably with the amino group, more preferably with the amino groups of lysine residue(s) of the VLP, and a further functional group which can react with the preferred second attachment site, i.e. a sulfhydryl group, preferably of cysteine(s) residue inherent of, or artificially added to the antigen, and optionally also made available for reaction by reduction. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company, and having one functional group reactive towards amino groups and one functional group reactive towards sulfhydryl groups. The above mentioned cross-linkers all lead to formation of an amide bond after reaction with the amino group and a thioether linkage with the sulfhydryl groups. Another class of cross-linkers suitable in the practice of the invention is characterized by the introduction of a disulfide linkage between the antigen and the VLP upon coupling. Preferred cross-linkers belonging to this class include, for example, SPDP and Sulfo-LC-SPDP (Pierce).

In a preferred embodiment, the composition of the invention further comprises a linker. In a further preferred embodiment said at least one antigen with said at least one second attachment site further comprises a linker, wherein said linker comprises said second attachment site, and wherein said linker is associated to said antigen by way of one peptide bond, and wherein preferably said linker is a cysteine. Engineering of a second attachment site onto the antigen is achieved by the association of a linker, preferably containing at least one amino acid suitable as second attachment site according to the disclosures of this invention. Therefore, in a preferred embodiment of the present invention, a linker is associated to the antigen by way of at least one covalent bond, preferably, by at least one, preferably one peptide bond. Preferably, the linker comprises, or alternatively consists of, the second attachment site. In a further preferred embodiment, the linker comprises a sulfhydryl group, preferably of a cysteine residue. In another preferred embodiment, the amino acid linker is a cysteine residue.

The selection of a linker will be dependent on the nature of the antigen, on its biochemical properties, such as pI, charge distribution and glycosylation. In general, flexible amino acid linkers are favored. In a further preferred embodiment of the present invention, the linker consists of amino acids, wherein further preferably the linker consists of at least one and at most 25, preferably at most 20, more preferably at most 15 amino acids. In an again preferred embodiment of the invention, the amino acid linker contains 1 to 10 amino acids. Preferred embodiments of the linker are selected from the group consisting of: (a) CGG; (b) N-terminal glycine linkers, preferably GCGGGG; (c) GGC; and (d) C-terminal glycine linkers, preferably GGGGCG. Further linkers useful for the invention are disclosed, for example, in WO2007/039552A1 (p. 32, paragraphs 111 and 112).

In a further preferred embodiment the linker is added to the N-terminus of the antigen.

In another preferred embodiment of the invention, the linker is added to the C-terminus of the antigen.

Preferred linkers according to this invention are glycine linkers (G)n further containing a cysteine residue as second attachment site. In general, glycine residues will be inserted between bulky amino acids and the cysteine to be used as second attachment site, to avoid potential steric hindrance of the bulkier amino acid in the coupling reaction. In a very preferred embodiment said linker with said second attachment site consists of GGC.

Thus, in a very preferred embodiment said at least one antigen with said at least one second attachment site comprises or preferably consists of any one of SEQ ID NOs 22, 25, 28 and 31, wherein most preferably said at least one antigen with said at least one second attachment site consists of SEQ ID NO:25.

Linking of the antigen to the VLP by using a hetero-bifunctional cross-linker according to the preferred methods described above, allows coupling of the antigen to the VLP in an oriented fashion. Other methods of linking the antigen to the VLP include methods wherein the antigen is cross-linked to the VLP, using the carbodiimide EDC, and NHS. The antigen may also be first thiolated through reaction, for example with SATA, SATP or iminothiolane. The antigen, after deprotection if required, may then be coupled to the VLP as follows. After separation of the excess thiolation reagent, the antigen is reacted with the VLP, previously activated with a hetero-bifunctional cross-linker comprising a cysteine reactive moiety, and therefore displaying at least one or several functional groups reactive towards cysteine residues, to which the thiolated antigen can react, such as described above. Optionally, low amounts of a reducing agent are included in the reaction mixture. In further methods, the antigen is attached to the VLP, using a homo-bifunctional cross-linker such as glutaraldehyde, DSG, BM[PEO]4, BS3, (Pierce) or other known homo-bifunctional cross-linkers with functional groups reactive towards amine groups or carboxyl groups of the VLP.

In other embodiments of the present invention, the composition comprises or alternatively consists essentially of a virus-like particle linked to the antigen via chemical interactions, wherein at least one of these interactions is not a covalent bond.

Linking of the VLP to the antigen can be effected by biotinylating the VLP and expressing the antigen as a streptavidin-fusion protein.

One or several antigen molecules can be attached to one subunit of RNA bacteriophage coat proteins, preferably through the exposed lysine residues of the coat proteins of RNA bacteriophage VLP, if sterically allowable. A specific feature of the VLPs of RNA bacteriophage and in particular of the Qβcoat protein VLP is thus the possibility to couple several antigens per subunit. This allows for the generation of a dense antigen array.

In very preferred embodiments of the invention, the antigen is linked via a cysteine residue, having been added to either the N-terminus or the C-terminus of, or a natural cysteine residue within the antigen, to lysine residues of coat proteins of the VLPs of RNA bacteriophage, and in particular to the coat protein of Qβ.

As described above, four lysine residues are exposed on the surface of the VLP of Qβ coat protein. Typically and preferably these residues are derivatized upon reaction with a cross-linker molecule. In the instance where not all of the exposed lysine residues can be coupled to an antigen, the lysine residues which have reacted with the cross-linker are left with a cross-linker molecule attached to the ε-amino group after the derivatization step. This leads to disappearance of one or several positive charges, which may be detrimental to the solubility and stability of the VLP. By replacing some of the lysine residues with arginines, as in the disclosed Qβ coat protein mutants, we prevent the excessive disappearance of positive charges since the arginine residues do not react with the preferred cross-linkers. Moreover, replacement of lysine residues by arginine residues may lead to more defined antigen arrays, as fewer sites are available for reaction to the antigen.

Accordingly, exposed lysine residues were replaced by arginines in the following Qβ coat protein mutants: Qβ-240 (Lys13-Arg; SEQ ID NO:14), Qβ-250 (Lys 2-Arg, Lys13-Arg; SEQ ID NO:16), Qβ-259 (Lys 2-Arg, Lys16-Arg; SEQ ID NO:18) and Qβ-251; (Lys16-Arg, SEQ ID NO:17). In a further embodiment, we disclose a Qβ mutant coat protein with one additional lysine residue Qβ-243 (Asn 10-Lys; SEQ ID NO:15), suitable for obtaining even higher density arrays of antigens.

In one preferred embodiment of the invention, the VLP of an RNA bacteriophage is recombinantly produced by a host and wherein said VLP is essentially free of host RNA, preferably host nucleic acids. In one further preferred embodiment, the composition further comprises at least one polyanionic macromolecule bound to, preferably packaged in or enclosed in, the VLP. In a still further preferred embodiment, the polyanionic macromolecule is polyglutamic acid and/or polyaspartic acid.

In another preferred embodiment, the composition further comprises at least one immunostimulatory substance bound to, preferably packaged in or enclosed in, the VLP. In a still further preferred embodiment, the immunostimulatory substance is a nucleic acid, preferably DNA, most preferably an unmethylated CpG containing oligonucleotide.

Essentially free of host RNA, preferably host nucleic acids: The term “essentially free of host RNA, preferably host nucleic acids” as used herein, refers to the amount of host RNA, preferably host nucleic acids, comprised by the VLP, which amount typically and preferably is less than 30 μg, preferably less than 20 μg, more preferably less than 10 μg, even more preferably less than 8 μg, even more preferably less than 6 μg, even more preferably less than 4 μg, most preferably less than 2 μg, per mg of the VLP. Host, as used within the afore-mentioned context, refers to the host in which the VLP is recombinantly produced. Conventional methods of determining the amount of RNA, preferably nucleic acids, are known to the skilled person in the art. The typical and preferred method to determine the amount of RNA, preferably nucleic acids, in accordance with the present invention is described in Example 17 of WO2006/037787A2. Identical, similar or analogous conditions are, typically and preferably, used for the determination of the amount of RNA, preferably nucleic acids, for inventive compositions comprising VLPs other than Qβ. The modifications of the conditions eventually needed are within the knowledge of the skilled person in the art. The numeric value of the amounts determined should typically and preferably be understood as comprising values having a deviation of ±10%, preferably having a deviation of ±5%, of the indicated numeric value.

Polyanionic macromolecule: The term “polyanionic macromolecule”, as used herein, refers to a molecule of high relative molecular mass which comprises repetitive groups of negative charge, the structure of which essentially comprises the multiple repetition of units derived, actually or conceptually, from molecules of low relative molecular mass. A polyanionic macromolecule should have a molecular weight of at least 2000 Dalton, more preferably of at least 3000 Dalton and even more preferably of at least 5000 Dalton. The term “polyanionic macromolecule” as used herein, typically and preferably refers to a molecule that is not capable of activating toll-like receptors. Thus, the term “polyanionic macromolecule” typically and preferably excludes Toll-like receptors ligands, and even more preferably furthermore excludes immunostimulatory substances such as Toll-like receptors ligands, immunostimulatory nucleic acids, and lipopolysacchrides (LPS). More preferably the term “polyanionic macromolecule” as used herein, refers to a molecule that is not capable of inducing cytokine production. Even more preferably the term “polyanionic macromolecule” excludes immunostimulatory substances. The term “immunostimulatory substance”, as used herein, refers to a molecule that is capable of inducing and/or enhancing immune response specifically against the antigen comprised in the present invention.

Host RNA, preferably host nucleic acids: The term “host RNA, preferably host nucleic acids” or the term “host RNA, preferably host nucleic acids, with secondary structure”, as used herein, refers to the RNA, or preferably nucleic acids, that are originally synthesized by the host. The RNA, preferably nucleic acids, may, however, undergo chemical and/or physical changes during the procedure of reducing or eliminating the amount of RNA, preferably nucleic acids, typically and preferably by way of the inventive methods, for example, the size of the RNA, preferably nucleic acids, may be shortened or the secondary structure thereof may be altered. However, even such resulting RNA or nucleic acids is still considered as host RNA, or host nucleic acids.

Methods to determine the amount of RNA and to reduce the amount of RNA comprised by the VLP have disclosed in WO2006/037787A2. Reducing or eliminating the amount of host RNA, preferably host nucleic, minimizes or reduces unwanted T cell responses, such as inflammatory T cell response and cytotoxic T cell response, and other unwanted side effects, such as fever, while maintaining strong antibody response specifically against dengue virus.

In one preferred embodiment, this invention provides a method of preparing the inventive compositions and VLP of an RNA bacteriophage the invention, wherein said VLP is recombinantly produced by a host and wherein said VLP is essentially free of host RNA, preferably host nucleic acids, comprising the steps of: a) recombinantly producing a virus-like particle (VLP) with at least one first attachment site by a host, wherein said VLP comprises coat proteins, variants or fragments thereof, of an RNA bacteriophage; b) disassembling said virus-like particle to said coat proteins, variants or fragments thereof, of said RNA bacteriophage; c) purifying said coat proteins, variants or fragments thereof; d) reassembling said purified coat proteins, variants or fragments thereof, of said RNA bacteriophage to a virus-like particle, wherein said virus-like particle is essentially free of host RNA, preferably host nucleic acids; and e) linking at least one antigen of the invention with at least one second attachment site to said VLP obtained from step d). In a further preferred embodiment, the reassembling of said purified coat proteins, variants or fragments thereof, is effected in the presence of at least one polyanionic macromolecule.

A further aspect of the invention is a composition for the treatment, amelioration and/or prevention of dengue fever, dengue hemorrhagic fever, and/or dengue shock syndrome, wherein said composition comprises (a) a virus-like particle with at least one first attachment site, wherein said virus-like particle is a virus-like particle of an RNA bacteriophage; and (b) at least one antigen with at least one second attachment site, wherein said at least one antigen is a dengue antigen, wherein said dengue antigen comprises at least position 9 to 99, position 9 to 109 or position 9 to 112 of domain III of the dengue virus envelope protein E; and wherein (a) and (b) are linked through said at least one first and said at least one second attachment site.

In one aspect, the invention provides a vaccine composition comprising or preferably consisting of a composition of the invention. Thus, the invention provides a vaccine composition comprising a composition, said composition comprising (a) a virus-like particle with at least one first attachment site, wherein said virus-like particle is a virus-like particle of an RNA bacteriophage; and (b) at least one antigen with at least one second attachment site, wherein said at least one antigen is a dengue antigen, wherein said dengue antigen comprises at least position 9 to 99, position 9 to 109 or position 9 to 112 of domain III of the dengue virus envelope protein E; and wherein (a) and (b) are linked through said at least one first and said at least one second attachment site.

An advantageous feature of the present invention is the high immunogenicity of the composition, even in the absence of adjuvants. Therefore, in a preferred embodiment, the vaccine composition is devoid of adjuvant. The absence of an adjuvant, furthermore, minimizes the occurrence of unwanted inflammatory T-cell responses representing a safety concern in the vaccination against self antigens. Thus, the administration of the vaccine composition to a patient will preferably occur without administering at least one adjuvant to the same patient prior to, simultaneously or after the administration of the vaccine composition.

However, when an adjuvant is administered, the administration of the at least one adjuvant may hereby occur prior to, simultaneously or after the administration of the inventive composition or of the vaccine composition. The term “adjuvant” as used herein refers to non-specific stimulators of the immune response or to substances that allow generation of a depot in the host which when combined with the composition, with the vaccine composition or with the pharmaceutical composition, respectively may provide for an even more enhanced immune response.

In a further embodiment, the vaccine composition further comprises at least one adjuvant.

In a further aspect, the invention provides a vaccine composition for the treatment, amelioration and/or prevention of dengue fever, dengue hemorrhagic fever, and/or dengue shock syndrome, wherein said vaccine composition comprises or preferably consists of a composition of the invention, and wherein preferably said vaccine composition is devoid of an adjuvant.

In a preferred embodiment the vaccine composition comprises or preferably consists of a first composition, of a second composition, of a third composition, and of a fourth composition, (i) wherein said first composition is a composition of the invention, wherein said domain III is domain III of the dengue virus envelope protein E of a dengue virus of serotype-1; (ii) wherein said second composition is a composition of the invention, wherein said domain III is domain III of the dengue virus envelope protein E of a dengue virus of serotype-2; (iii) wherein said third composition is a composition of the invention, wherein said domain III is domain III of the dengue virus envelope protein E of a dengue virus of serotype-3; and (iv) wherein said fourth composition is a composition of the invention, wherein said domain III is domain III of the dengue virus envelope protein E of a dengue virus of serotype-4.

In a preferred embodiment, the present invention provides for a composition comprising (i) a first composition, (ii) a second composition (iii) a third composition, and (iv) a fourth composition. In a preferred embodiment of the present invention, the first composition is a composition of the invention comprising (a) a virus-like particle of RNA bacteriophage Qβ with at least one first attachment site, wherein said virus-like particle of RNA bacteriophage Qβ comprises one or more recombinant coat proteins having the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-1, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-1. In another preferred embodiment of the present invention, said dengue antigen comprises, or preferably consists of, at least position 9 to 112 of domain III of the dengue virus envelope protein E of a dengue virus of serotype-1. In a further preferred embodiment of the present invention, said dengue antigen comprises, or preferably consists of, at least position 1 to 113 of domain III of the dengue virus envelope protein E of a dengue virus of serotype-1.

In a preferred embodiment of the present invention, the first composition is a composition of the invention comprising (a) a virus-like particle of RNA bacteriophage Qβ with at least one first attachment site, wherein said virus-like particle of RNA bacteriophage Qβ comprises one or more recombinant coat proteins consisting of the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-1, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-1. In another preferred embodiment of the present invention, said dengue antigen comprises, or preferably consists of, at least position 9 to 112 of domain III of the dengue virus envelope protein E of a dengue virus of serotype-1. In a further preferred embodiment of the present invention, said dengue antigen comprises, or preferably consists of, at least position 1 to 113 of domain III of the dengue virus envelope protein E of a dengue virus of serotype-1.

In a preferred embodiment of the present invention, the second composition is a composition of the invention comprising (a) a virus-like particle of RNA bacteriophage Qβ with at least one first attachment site, wherein said virus-like particle of RNA bacteriophage Qβ comprises one or more recombinant coat proteins having the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-2, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-2. In another preferred embodiment of the present invention, said dengue antigen comprises, or preferably consists of, at least position 9 to 112 of domain III of the dengue virus envelope protein E of a dengue virus of serotype-2. In a further preferred embodiment of the present invention, said dengue antigen comprises, or preferably consists of, at least position 1 to 113 of domain III of the dengue virus envelope protein E of a dengue virus of serotype-2.

In a preferred embodiment of the present invention, the second composition is a composition of the invention comprising (a) a virus-like particle of RNA bacteriophage Qβ with at least one first attachment site, wherein said virus-like particle of RNA bacteriophage Qβ comprises one or more recombinant coat proteins consisting of the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-2, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-2. In another preferred embodiment of the present invention, said dengue antigen comprises, or preferably consists of, at least position 9 to 112 of domain III of the dengue virus envelope protein E of a dengue virus of serotype-2. In a further preferred embodiment of the present invention, said dengue antigen comprises, or preferably consists of, at least position 1 to 113 of domain III of the dengue virus envelope protein E of a dengue virus of serotype-2.

In a preferred embodiment of the present invention, the third composition is a composition of the invention comprising (a) a virus-like particle of RNA bacteriophage Qβ with at least one first attachment site, wherein said virus-like particle of RNA bacteriophage Qβ comprises one or more recombinant coat proteins having the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-3, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-3. In another preferred embodiment of the present invention, said dengue antigen comprises, or preferably consists of, at least position 9 to 112 of domain III of the dengue virus envelope protein E of a dengue virus of serotype-3. In a further preferred embodiment of the present invention, said dengue antigen comprises, or preferably consists of, at least position 1 to 113 of domain III of the dengue virus envelope protein E of a dengue virus of serotype-3.

In a preferred embodiment of the present invention, the third composition is a composition of the invention comprising (a) a virus-like particle of RNA bacteriophage Qβ with at least one first attachment site, wherein said virus-like particle of RNA bacteriophage Qβ comprises one or more recombinant coat proteins consisting of the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-3, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-3. In another preferred embodiment of the present invention, said dengue antigen comprises, or preferably consists of, at least position 9 to 112 of domain III of the dengue virus envelope protein E of a dengue virus of serotype-3. In a further preferred embodiment of the present invention, said dengue antigen comprises, or preferably consists of, at least position 1 to 113 of domain III of the dengue virus envelope protein E of a dengue virus of serotype-3.

In a preferred embodiment of the present invention, the fourth composition is a composition of the invention comprising (a) a virus-like particle of RNA bacteriophage Qβ with at least one first attachment site, wherein said virus-like particle of RNA bacteriophage Qβ comprises one or more recombinant coat proteins having the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-4, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-4. In another preferred embodiment of the present invention, said dengue antigen comprises, or preferably consists of, at least position 9 to 112 of domain III of the dengue virus envelope protein E of a dengue virus of serotype-4. In a further preferred embodiment of the present invention, said dengue antigen comprises, or preferably consists of, at least position 1 to 113 of domain III of the dengue virus envelope protein E of a dengue virus of serotype-4.

In a preferred embodiment of the present invention, the fourth composition is a composition of the invention comprising (a) a virus-like particle of RNA bacteriophage Qβ with at least one first attachment site, wherein said virus-like particle of RNA bacteriophage Qβ comprises one or more recombinant coat proteins consisting of the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-4, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-4. In another preferred embodiment of the present invention, said dengue antigen comprises, or preferably consists of, at least position 9 to 112 of domain III of the dengue virus envelope protein E of a dengue virus of serotype-4. In a further preferred embodiment of the present invention, said dengue antigen comprises, or preferably consists of, at least position 1 to 113 of domain III of the dengue virus envelope protein E of a dengue virus of serotype-4.

In a preferred embodiment of the present invention, the first composition is a composition of the invention comprising (a) a virus-like particle of RNA bacteriophage AP205 with at least one first attachment site, wherein said virus-like particle of RNA bacteriophage AP205 comprises one or more recombinant coat proteins having the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-1, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-1. In another preferred embodiment of the present invention, said dengue antigen comprises, or preferably consists of, at least position 9 to 112 of domain III of the dengue virus envelope protein E of a dengue virus of serotype-1. In a further preferred embodiment of the present invention, said dengue antigen comprises, or preferably consists of, at least position 1 to 113 of domain III of the dengue virus envelope protein E of a dengue virus of serotype-1.

In a preferred embodiment of the present invention, the first composition is a composition of the invention comprising (a) a virus-like particle of RNA bacteriophage AP205 with at least one first attachment site, wherein said virus-like particle of RNA bacteriophage AP205 comprises one or more recombinant coat proteins consisting of the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-1, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-1. In another preferred embodiment of the present invention, said dengue antigen comprises, or preferably consists of, at least position 9 to 112 of domain III of the dengue virus envelope protein E of a dengue virus of serotype-1. In a further preferred embodiment of the present invention, said dengue antigen comprises, or preferably consists of, at least position 1 to 113 of domain III of the dengue virus envelope protein E of a dengue virus of serotype-1.

In a preferred embodiment of the present invention, the second composition is a composition of the invention comprising (a) a virus-like particle of RNA bacteriophage AP205 with at least one first attachment site, wherein said virus-like particle of RNA bacteriophage AP205 comprises one or more recombinant coat proteins having the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-2, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-2. In another preferred embodiment of the present invention, said dengue antigen comprises, or preferably consists of, at least position 9 to 112 of domain III of the dengue virus envelope protein E of a dengue virus of serotype-2. In a further preferred embodiment of the present invention, said dengue antigen comprises, or preferably consists of, at least position 1 to 113 of domain III of the dengue virus envelope protein E of a dengue virus of serotype-2.

In a preferred embodiment of the present invention, the second composition is a composition of the invention comprising (a) a virus-like particle of RNA bacteriophage AP205 with at least one first attachment site, wherein said virus-like particle of RNA bacteriophage AP205 comprises one or more recombinant coat proteins consisting of the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-2, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-2. In another preferred embodiment of the present invention, said dengue antigen comprises, or preferably consists of, at least position 9 to 112 of domain III of the dengue virus envelope protein E of a dengue virus of serotype-2. In a further preferred embodiment of the present invention, said dengue antigen comprises, or preferably consists of, at least position 1 to 113 of domain III of the dengue virus envelope protein E of a dengue virus of serotype-2.

In a preferred embodiment of the present invention, the third composition is a composition of the invention comprising (a) a virus-like particle of RNA bacteriophage AP205 with at least one first attachment site, wherein said virus-like particle of RNA bacteriophage AP205 comprises one or more recombinant coat proteins having the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-3, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-3. In another preferred embodiment of the present invention, said dengue antigen comprises, or preferably consists of, at least position 9 to 112 of domain III of the dengue virus envelope protein E of a dengue virus of serotype-3. In a further preferred embodiment of the present invention, said dengue antigen comprises, or preferably consists of, at least position 1 to 113 of domain III of the dengue virus envelope protein E of a dengue virus of serotype-3.

In a preferred embodiment of the present invention, the third composition is a composition of the invention comprising (a) a virus-like particle of RNA bacteriophage AP205 with at least one first attachment site, wherein said virus-like particle of RNA bacteriophage AP205 comprises one or more recombinant coat proteins consisting of the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-3, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-3. In another preferred embodiment of the present invention, said dengue antigen comprises, or preferably consists of, at least position 9 to 112 of domain III of the dengue virus envelope protein E of a dengue virus of serotype-3. In a further preferred embodiment of the present invention, said dengue antigen comprises, or preferably consists of, at least position 1 to 113 of domain III of the dengue virus envelope protein E of a dengue virus of serotype-3.

In a preferred embodiment of the present invention, the fourth composition is a composition of the invention comprising (a) a virus-like particle of RNA bacteriophage AP205 with at least one first attachment site, wherein said virus-like particle of RNA bacteriophage AP205 comprises one or more recombinant coat proteins having the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-4, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-4. In another preferred embodiment of the present invention, said dengue antigen comprises, or preferably consists of, at least position 9 to 112 of domain III of the dengue virus envelope protein E of a dengue virus of serotype-4. In a further preferred embodiment of the present invention, said dengue antigen comprises, or preferably consists of, at least position 1 to 113 of domain III of the dengue virus envelope protein E of a dengue virus of serotype-4.

In a preferred embodiment of the present invention, the fourth composition is a composition of the invention comprising (a) a virus-like particle of RNA bacteriophage AP205 with at least one first attachment site, wherein said virus-like particle of RNA bacteriophage AP205 comprises one or more recombinant coat proteins consisting of the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, at least position 9 to 99 of domain III of the dengue virus envelope protein E of a dengue virus of serotype-4, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein said hetero-bifunctional cross-linker is SMPH. In a further preferred embodiment of the present invention, said dengue antigen comprises, or preferably consists of, at least position 9 to 109 of domain III of the dengue virus envelope protein E of a dengue virus of serotype-4. In another preferred embodiment of the present invention, said dengue antigen comprises, or preferably consists of, at least position 9 to 112 of domain III of the dengue virus envelope protein E of a dengue virus of serotype-4. In a further preferred embodiment of the present invention, said dengue antigen comprises, or preferably consists of, at least position 1 to 113 of domain III of the dengue virus envelope protein E of a dengue virus of serotype-4.

In a preferred embodiment, the present invention provides for a composition comprising, or preferably consisting of, (i) a first composition, (ii) a second composition (iii) a third composition, and (iv) a fourth composition, wherein each of said first, second, third and fourth composition are present in equal amounts.

In a further preferred embodiment, the present invention provides for a composition comprising, or preferably consisting of, (i) a first composition, (ii) a second composition (iii) a third composition, and (iv) a fourth composition, wherein said first composition is present in an amount W, said second composition is present in an amount X, said third composition is present in an amount Y, and wherein said fourth composition is present in an amount Z leading to a ratio of W:X:Y:Z, wherein each of said W, X, Y, Z ranges from 0.1 to 10, preferably from 0.25 to 5, and wherein further preferably each of said W, X, Y, Z ranges from 0.5 to 5, again preferably from 0.6 to 4.8, and wherein further preferably each of said W, X, Y, Z ranges from 0.7 to 4.2, preferably from 0.8 to 3.2, and wherein further preferably each of said W, X,Y, Z ranges from 0.9 to 2.7, preferably from 0.9 to 1.8, further preferably from 0.75 to 1.25. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment, the present invention provides for a composition comprising, or preferably consisting of, (i) a first composition, (ii) a second composition (iii) a third composition, and (iv) a fourth composition, wherein said first composition is present in an amount W, said second composition is present in an amount X, said third composition is present in an amount Y, and wherein said fourth composition is present in an amount Z leading to a ratio of W:X:Y:Z, wherein said ratio is selected from the group consisting of 1:1:1:1, 1:1:1:2, 1:1:1:3, 1:1:1:4, 1:1:1:5, 1:1:1:6, 1:1:1:7, 1:1:1:8, 1:1:1:9, 1:1:1:10 and 0.5:1.5:2:5. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment, the present invention provides for a composition comprising, or preferably consisting of, (i) a first composition, (ii) a second composition (iii) a third composition, and (iv) a fourth composition, wherein said first composition is present in an amount W, said second composition is present in an amount X, said third composition is present in an amount Y, and wherein said fourth composition is present in an amount Z leading to a ratio of W:X:Y:Z, wherein said ratio is 1.1:1:1. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment, the present invention provides for a composition comprising, or preferably consisting of, (i) a first composition, (ii) a second composition (iii) a third composition, and (iv) a fourth composition, wherein said first composition is present in an amount W, said second composition is present in an amount X, said third composition is present in an amount Y, and wherein said fourth composition is present in an amount Z leading to a ratio of W:X:Y:Z, wherein said ratio is 1.1:1:5. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment, the present invention provides for a composition comprising, or preferably consisting of, (i) a first composition, (ii) a second composition (iii) a third composition, and (iv) a fourth composition, wherein said first composition is present in an amount W, said second composition is present in an amount X, said third composition is present in an amount Y, and wherein said fourth composition is present in an amount Z leading to a ratio of W:X:Y:Z, wherein said ratio is 1.1:1:20. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment the vaccine composition comprises or preferably consists of a first composition, of a second composition, of a third composition, and of a fourth composition, (i) wherein said first composition is a composition of the invention, wherein said dengue antigen comprises or preferably consists of SEQ ID NO:21; (ii) wherein said second composition is a composition of the invention, wherein said dengue antigen comprises or preferably consists of SEQ ID NO:24; (iii) wherein said third composition is a composition of the invention, wherein said dengue antigen comprises or preferably consists of SEQ ID NO:27; and (iv) wherein said fourth composition is a composition of the invention, wherein said dengue antigen comprises or preferably consists of SEQ ID NO:30 or SEQ ID NO:32.

In a further preferred embodiment the vaccine composition comprises or preferably consists of a first composition, of a second composition, of a third composition, and of a fourth composition, (i) wherein said first composition is a composition of the invention, wherein said at least one antigen with said at least one second attachment site comprises or preferably consists of SEQ ID NO:22; (ii) wherein said second composition is a composition of the invention, wherein said at least one antigen with said at least one second attachment site comprises or preferably consists of SEQ ID NO:25; (iii) wherein said third composition is a composition of the invention, wherein said at least one antigen with said at least one second attachment site comprises or preferably consists of SEQ ID NO:28; and (iv) wherein said fourth composition is a composition of the invention, wherein said at least one antigen with said at least one second attachment site comprises or preferably consists of SEQ ID NO:31.

In one embodiment, the present invention provides a method of inducing an immune response to the domain III of the dengue virus envelope protein E of a dengue virus of serotype-1, serotype-2, serotype-3 and serotype-4 in mammals, involving administering to the said mammal a therapeutically effective amount of the composition comprising, or preferably consisting of, (i) a first composition, (ii) a second composition, (iii) a third composition, and (iv) a fourth composition, wherein said first composition (i) comprises a virus-like particle of an RNA bacteriophage with at least one first attachment site and at least one antigen with at least one second attachment site, wherein said at least one antigen is a dengue antigen comprising, or preferably consisting of, at least position 9 to 99 of domain III of the dengue virus envelope protein E of a dengue virus of serotype-1; and wherein said second composition (ii) comprises a virus-like particle of an RNA bacteriophage with at least one first attachment site and at least one antigen with at least one second attachment site, wherein said at least one antigen is a dengue antigen comprising, or preferably consisting of, at least position 9 to 99 of domain III of the dengue virus envelope protein E of a dengue virus of serotype-2; and wherein said third composition (iii) comprises a virus-like particle of an RNA bacteriophage with at least one first attachment site and at least one antigen with at least one second attachment site, wherein said at least one antigen is a dengue antigen comprising, or preferably consisting of, at least position 9 to 99 of domain III of the dengue virus envelope protein E of a dengue virus of serotype-3; and wherein said fourth composition (iv) comprises a virus-like particle of an RNA bacteriophage with at least one first attachment site and at least one antigen with at least one second attachment site, wherein said at least one antigen is a dengue antigen comprising, or preferably consisting of, at least position 9 to 99 of domain III of the dengue virus envelope protein E of a dengue virus of serotype-4; and wherein said virus-like particles of an RNA bacteriophage and said at least one antigen of each of said first composition, said second composition, said third composition and said fourth composition are linked through said at least one first and said at least one second attachment site.

In a preferred embodiment, the present invention provides for a composition comprising (i) a first composition, (ii) a second composition (iii) a third composition, and (iv) a fourth composition, wherein said first composition (i) comprises (a) a virus-like particle of RNA bacteriophage Qβ with at least one first attachment site, wherein said virus-like particle of RNA bacteriophage Qβ comprises one or more recombinant coat proteins having, preferably consisting of, the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, SEQ ID NO:21, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said second composition (ii) comprises (a) a virus-like particle of RNA bacteriophage Qβ with at least one first attachment site, wherein said virus-like particle of RNA bacteriophage Qβ comprises one or more recombinant coat proteins having, preferably consisting of, the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, SEQ ID NO:24, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said third composition (iii) comprises (a) a virus-like particle of RNA bacteriophage Qβ with at least one first attachment site, wherein said virus-like particle of RNA bacteriophage Qβ comprises one or more recombinant coat proteins having, preferably consisting of, the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, SEQ ID NO:27, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said fourth composition (iv) is a composition of the invention comprising (a) a virus-like particle of RNA bacteriophage Qβ with at least one first attachment site, wherein said virus-like particle of RNA bacteriophage Qβ comprises one or more recombinant coat proteins having, preferably consisting of, the amino acid sequence as set forth in SEQ ID NO:1, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, SEQ ID NO:30, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. Preferably each of said first, second, third and fourth composition are present in equal amounts. In a further preferred embodiment, said first composition is present in an amount W, said second composition is present in an amount X, said third composition is present in an amount Y, and wherein said fourth composition is present in an amount Z leading to a ratio of W:X:Y:Z, wherein each of said W, X, Y, Z ranges from 0.1 to 10, preferably from 0.25 to 5, and wherein further preferably each of said W, X, Y, Z ranges from 0.5 to 5, again preferably from 0.6 to 4.8, and wherein further preferably each of said W, X, Y, Z ranges from 0.7 to 4.2, preferably from 0.8 to 3.2, and wherein further preferably each of said W, X,Y, Z ranges from 0.9 to 2.7, preferably from 0.9 to 1.8, further preferably from 0.75 to 1.25. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is selected from the group consisting of 1:1:1:1, 1:1:1:2, 1:1:1:3, 1:1:1:4, 1:1:1:5, 1:1:1:6, 1:1:1:7, 1:1:1:8, 1:1:1:9, 1:1:1:10 and 0.5:1.5:2:5. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:1. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:5. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:20. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment of the present invention, the composition comprises (i) a first composition, (ii) a second composition (iii) a third composition, and (iv) a fourth composition, wherein said first composition (i) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, SEQ ID NO:21, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said second composition (ii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, SEQ ID NO:24, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said third composition (iii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, SEQ ID NO:27, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said fourth composition (iv) is a composition of the invention comprising (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen comprises, or preferably consists of, SEQ ID NO:30, and wherein said at least one antigen with said at least one second attachment site further comprises a linker, wherein said linker comprises said second attachment site, and wherein said linker is associated to the C-terminus of said antigen by way of one peptide bond, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. Preferably each of said first, second, third and fourth composition are present in equal amounts. In a further preferred embodiment, said first composition is present in an amount W, said second composition is present in an amount X, said third composition is present in an amount Y, and wherein said fourth composition is present in an amount Z leading to a ratio of W:X:Y:Z, wherein each of said W, X,Y, Z ranges from 0.1 to 10, preferably from 0.25 to 5, and wherein further preferably each of said W, X,Y, Z ranges from 0.5 to 5, again preferably from 0.6 to 4.8, and wherein further preferably each of said W, X,Y, Z ranges from 0.7 to 4.2, preferably from 0.8 to 3.2, and wherein further preferably each of said W, X,Y, Z ranges from 0.9 to 2.7, preferably from 0.9 to 1.8, further preferably from 0.75 to 1.25. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is selected from the group consisting of 1:1:1:1, 1:1:1:2, 1:1:1:3, 1:1:1:4, 1:1:1:5, 1:1:1:6, 1:1:1:7, 1:1:1:8, 1:1:1:9, 1:1:1:10 and 0.5:1.5:2:5. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:1. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:5. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:20. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment of the present invention, the composition comprises (i) a first composition, (ii) a second composition (iii) a third composition, and (iv) a fourth composition, wherein said first composition (i) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:33, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said second composition (ii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:34, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said third composition (iii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO: 35, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said fourth composition (iv) is a composition of the invention comprising (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:36, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. Preferably each of said first, second, third and fourth composition are present in equal amounts. In a further preferred embodiment, said first composition is present in an amount W, said second composition is present in an amount X, said third composition is present in an amount Y, and wherein said fourth composition is present in an amount Z leading to a ratio of W:X:Y:Z, wherein each of said W, X,Y, Z ranges from 0.1 to 10, preferably from 0.25 to 5, and wherein further preferably each of said W, X,Y, Z ranges from 0.5 to 5, again preferably from 0.6 to 4.8, and wherein further preferably each of said W, X,Y, Z ranges from 0.7 to 4.2, preferably from 0.8 to 3.2, and wherein further preferably each of said W, X, Y, Z ranges from 0.9 to 2.7, preferably from 0.9 to 1.8, further preferably from 0.75 to 1.25. Preferably each of said W, X, Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is selected from the group consisting of 1:1:1:1, 1:1:1:2, 1:1:1:3, 1:1:1:4, 1:1:1:5, 1:1:1:6, 1:1:1:7, 1:1:1:8, 1:1:1:9, 1:1:1:10 and 0.5:1.5:2:5. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:1. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:5. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:20. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment of the present invention, the composition comprises (i) a first composition, (ii) a second composition (iii) a third composition, and (iv) a fourth composition, wherein said first composition (i) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:33, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said second composition (ii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:34, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said third composition (iii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO: 35, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said fourth composition (iv) is a composition of the invention comprising (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:37, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. Preferably each of said first, second, third and fourth composition are present in equal amounts. In a further preferred embodiment, said first composition is present in an amount W, said second composition is present in an amount X, said third composition is present in an amount Y, and wherein said fourth composition is present in an amount Z leading to a ratio of W:X:Y:Z, wherein each of said W, X,Y, Z ranges from 0.1 to 10, preferably from 0.25 to 5, and wherein further preferably each of said W, X,Y, Z ranges from 0.5 to 5, again preferably from 0.6 to 4.8, and wherein further preferably each of said W, X,Y, Z ranges from 0.7 to 4.2, preferably from 0.8 to 3.2, and wherein further preferably each of said W, X,Y, Z ranges from 0.9 to 2.7, preferably from 0.9 to 1.8, further preferably from 0.75 to 1.25. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is selected from the group consisting of 1:1:1:1, 1:1:1:2, 1:1:1:3, 1:1:1:4, 1:1:1:5, 1:1:1:6, 1:1:1:7, 1:1:1:8, 1:1:1:9, 1:1:1:10 and 0.5:1.5:2:5. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:1. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:5. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:20. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment of the present invention, the composition comprises (i) a first composition, (ii) a second composition (iii) a third composition, and (iv) a fourth composition, wherein said first composition (i) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:33, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said second composition (ii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:34, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said third composition (iii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO: 35, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said fourth composition (iv) is a composition of the invention comprising (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:38, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. Preferably each of said first, second, third and fourth composition are present in equal amounts. In a further preferred embodiment, said first composition is present in an amount W, said second composition is present in an amount X, said third composition is present in an amount Y, and wherein said fourth composition is present in an amount Z leading to a ratio of W:X:Y:Z, wherein each of said W, X,Y, Z ranges from 0.1 to 10, preferably from 0.25 to 5, and wherein further preferably each of said W, X,Y, Z ranges from 0.5 to 5, again preferably from 0.6 to 4.8, and wherein further preferably each of said W, X,Y, Z ranges from 0.7 to 4.2, preferably from 0.8 to 3.2, and wherein further preferably each of said W, X,Y, Z ranges from 0.9 to 2.7, preferably from 0.9 to 1.8, further preferably from 0.75 to 1.25. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is selected from the group consisting of 1:1:1:1, 1:1:1:2, 1:1:1:3, 1:1:1:4, 1:1:1:5, 1:1:1:6, 1:1:1:7, 1:1:1:8, 1:1:1:9, 1:1:1:10 and 0.5:1.5:2:5. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:1. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:5. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:20. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment of the present invention, the composition comprises (i) a first composition, (ii) a second composition (iii) a third composition, and (iv) a fourth composition, wherein said first composition (i) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:33, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said second composition (ii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:34, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said third composition (iii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO: 35, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said fourth composition (iv) is a composition of the invention comprising (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:39, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. Preferably each of said first, second, third and fourth composition are present in equal amounts. In a further preferred embodiment, said first composition is present in an amount W, said second composition is present in an amount X, said third composition is present in an amount Y, and wherein said fourth composition is present in an amount Z leading to a ratio of W:X:Y:Z, wherein each of said W, X,Y, Z ranges from 0.1 to 10, preferably from 0.25 to 5, and wherein further preferably each of said W, X,Y, Z ranges from 0.5 to 5, again preferably from 0.6 to 4.8, and wherein further preferably each of said W, X,Y, Z ranges from 0.7 to 4.2, preferably from 0.8 to 3.2, and wherein further preferably each of said W, X,Y, Z ranges from 0.9 to 2.7, preferably from 0.9 to 1.8, further preferably from 0.75 to 1.25. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is selected from the group consisting of 1:1:1:1, 1:1:1:2, 1:1:1:3, 1:1:1:4, 1:1:1:5, 1:1:1:6, 1:1:1:7, 1:1:1:8, 1:1:1:9, 1:1:1:10 and 0.5:1.5:2:5. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:1. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:5. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:20. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E.

In a further preferred embodiment of the present invention, the composition comprises (i) a first composition, (ii) a second composition (iii) a third composition, and (iv) a fourth composition, wherein said first composition (i) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:33, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said second composition (ii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:34, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said third composition (iii) comprises (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO: 35, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH, and wherein said fourth composition (iv) is a composition of the invention comprising (a) a virus-like particle of RNA bacteriophage AP205 with at least one first attachment site, wherein said virus-like particle of RNA bacteriophage AP205 comprises one or more recombinant coat proteins having, preferably consisting of, the amino acid sequence as set forth in SEQ ID NO:19, and wherein said first attachment site is a lysine residue and wherein said lysine residue is part of said recombinant coat protein; and (b) at least one antigen with at least one second attachment site, wherein said at least one antigen is a dengue antigen, and wherein said dengue antigen with said second attachment site comprises, or preferably consists of, SEQ ID NO:40, wherein said second attachment site is a cysteine residue, and wherein said cysteine residue is artificially added, and wherein said first attachment site associates with said second attachment site through a hetero-bifunctional cross-linker, wherein preferably said hetero-bifunctional cross-linker is SMPH. Preferably each of said first, second, third and fourth composition are present in equal amounts. In a further preferred embodiment, said first composition is present in an amount W, said second composition is present in an amount X, said third composition is present in an amount Y, and wherein said fourth composition is present in an amount Z leading to a ratio of W:X:Y:Z, wherein each of said W, X,Y, Z ranges from 0.1 to 10, preferably from 0.25 to 5, and wherein further preferably each of said W, X,Y, Z ranges from 0.5 to 5, again preferably from 0.6 to 4.8, and wherein further preferably each of said W, X,Y, Z ranges from 0.7 to 4.2, preferably from 0.8 to 3.2, and wherein further preferably each of said W, X,Y, Z ranges from 0.9 to 2.7, preferably from 0.9 to 1.8, further preferably from 0.75 to 1.25. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is selected from the group consisting of 1:1:1:1, 1:1:1:2, 1:1:1:3, 1:1:1:4, 1:1:1:5, 1:1:1:6, 1:1:1:7, 1:1:1:8, 1:1:1:9, 1:1:1:10 and 0.5:1.5:2:5. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:1. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:5. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E. In a further preferred embodiment, said first composition is present in an amount W, said second composition is present in an amount X, said third composition is present in an amount Y, and wherein said fourth composition is present in an amount Z leading to a ratio of W:X:Y:Z, wherein said ratio is 1.1:1:20. Preferably each of said W, X,Y, Z are selected in a manner that said composition is capable of inducing a balanced immune response against all four serotypes, preferably against serotype-1, serotype-2, serotype-3 and serotype-4 of domain III of the dengue virus envelope protein E.

When the composition and/or the vaccine composition of the invention is administered to an individual, it may be in a form which contains salts, buffers, adjuvants, or other substances which are desirable for improving the efficacy of the conjugate. Examples of materials suitable for use in preparation of vaccine compositions or pharmaceutical compositions are provided in numerous sources including Remington's Pharmaceutical Sciences (Osol, A, ed., Mack Publishing Co., (1990)). This includes sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

The vaccine compositions of the invention are said to be “pharmaceutically acceptable” if their administration can be tolerated by a recipient individual, preferably by a human. Further, the vaccine compositions of the invention are administered in a “therapeutically effective amount” (i.e., an amount that produces a desired physiological effect). The nature or type of immune response is not a limiting factor of this disclosure. Without the intention to limit the present invention by the following mechanistic explanation, the inventive vaccine compositions might induce antibodies which bind to the envelope protein E of dengue virus, and, thus, is capable of neutralizing the virus in vivo.

The invention further provides a pharmaceutical composition comprising: (1) a vaccine composition of the invention; and (2) a pharmaceutically acceptable carrier. More specifically, the invention provides a pharmaceutical composition, said pharmaceutical composition comprising (1) (a) a virus-like particle with at least one first attachment site, wherein said virus-like particle is a virus-like particle of an RNA bacteriophage; and (b) at least one antigen with at least one second attachment site, wherein said at least one antigen is a dengue antigen, wherein said dengue antigen comprises at least position 9 to 99, position 9 to 109 or position 9 to 112 of domain III of the dengue virus envelope protein E; and wherein (a) and (b) are linked through said at least one first and said at least one second attachment site; and (2) a pharmaceutically acceptable carrier.

The invention further provides a pharmaceutical composition for the treatment, amelioration and/or prevention of dengue fever, dengue hemorrhagic fever, and/or dengue shock syndrome, said pharmaceutical composition comprising (1) a vaccine composition of the invention, and (2) a pharmaceutically acceptable carrier.

The invention further provides a method for the treatment, amelioration and/or prevention of dengue fever, dengue hemorrhagic fever, and/or dengue shock syndrome, said method comprising administering a composition, a vaccine composition or a pharmaceutical composition of the invention to an animal, preferably to a human. Thus, the invention provides a method for the treatment, amelioration and/or prevention of dengue fever, dengue hemorrhagic fever, and/or dengue shock syndrome, said method comprising administering a composition to an animal, preferably to a human, said composition comprising (a) a virus-like particle with at least one first attachment site, wherein said virus-like particle is a virus-like particle of an RNA bacteriophage; and (b) at least one antigen with at least one second attachment site, wherein said at least one antigen is a dengue antigen, wherein said dengue antigen comprises at least position 9 to 99 position 9 to 109 or position 9 to 112 of domain III of the dengue virus envelope protein E; and wherein (a) and (b) are linked through said at least one first and said at least one second attachment site.

With respect to the methods of the invention, said composition, said vaccine and/or said pharmaceutical composition is preferably administered to said animal, more preferably to said human, in an immunologically effective amount.

In one embodiment, the compositions, vaccine compositions and/or pharmaceutical compositions are administered to said animal, preferably to said human by injection, infusion, inhalation, oral administration, or other suitable physical methods. In a preferred embodiment, the compositions, vaccine compositions and/or pharmaceutical compositions are administered to said animal, preferably to said human, intramuscularly, intravenously, transmucosally, transdermally, intranasally, intraperitoneally, subcutaneously, or directly into the lymph node.

A further aspect of the invention is the use of the compositions, of the vaccine compositions and/or of the pharmaceutical compositions described herein for the treatment, amelioration and/or prevention of dengue fever, dengue hemorrhagic fever, and/or dengue shock syndrome.

A further aspect of the invention is the use of the compositions, the vaccine compositions and/or of the pharmaceutical compositions described herein for the manufacture of a medicament for the treatment, amelioration and/or prevention of dengue fever, dengue hemorrhagic fever, and/or dengue shock syndrome, preferably in an animal, most preferably in a human.

It is to be understood that all technical features and embodiments described herein, in particular those described for the compositions of the invention, may be applied to all aspects of the invention, especially to the vaccine compositions, pharmaceutical compositions, methods and uses, alone or in any possible combination.

### EXAMPLES

### Example 1

### Cloning of Envelope Protein E Domain III

Total gene synthesis of a nucleic acid sequence encoding dengue serotype-2 envelope protein E domain III of strain Thailand/NGS-C/1944 (Swissprot: P14340) was performed using overlapping oligonucleotides and standard PCR assembly methods to give a fragment with an Nde I restriction site at the 5′-end and an Xho I restriction site at the 3′-end, resulting in SEQ ID NO:23.

Nucleic sequences encoding dengue envelope protein E domain III of serotype-1 (strain Reunion 305/04; Swissprot: A0S5S5), of serotype-3 (strain Singapore/8120/1995; Swissprot: Q5UB51), and of serotype-4 (strain MY01-23314; Swissprot: Q8B0G5) were synthesized with an Nde I restriction site at the 5′-end and an Xho I restriction site at the 3′-end, whereby a codon optimization for E. coli was performed (service by Geneart, Regensburg, Germany). The resulting nucleic acid sequences were SEQ ID NO:20 (serotype-1), SEQ ID NO:26 (serotype-3), and SEQ ID NO:29 (serotype-4). The Nde I/Xho I fragments of all 4 serotypes were subcloned into the corresponding sites of an expression vector derivative of pET-42a(+) (Novagen, Dietikon, Switzerland) to give the following vectors: pET42T_DV1-E-DIII, pET42T_DV2-E-DIII, pET42T_DV3-E-DIII, pET42T_DV4-E-DIII. This did add to the domain III C-terminally a LE-linker (Xho I site), a His₆-tag, a GG-linker and a cysteine at C-terminus (DEN-1: SEQ ID NO22 (domain III of the dengue virus envelope protein E of serotype-1); DEN-2: SEQ ID NO:25 (domain III of the dengue virus envelope protein E of serotype-2); DEN-3: SEQ ID NO:28 (domain III of the dengue virus envelope protein E of serotype-3); DEN-4: SEQ ID NO:31 (domain III of the dengue virus envelope protein E of serotype-4)).

### Example 2

### Expression of Envelope Protein E Domain III

Competent E. coli BL21 (DE3) were transformed with the expression vector plasmids described in Example 1. A single colony of a kanamycin containing agar plate was grown overnight in liquid culture and used to inoculate 1 l of kanamycin containing LB medium. The culture was grown at 37° C. with 150 rpm to OD₆₀₀ nm=1.0. Expression was induced with 1 mM IPTG. Bacteria were harvested after an overnight induction at 37° C. by centrifuging for 15 minutes at 6000 rpm at 4° C.

### Example 3

### Purification of Envelope Protein E Domain III

The cell pellets from Example 2 were resuspended in 25 ml buffer (PBS, 10 mM MgCl₂, 0.25% Triton X-100, pH 7.2) and sonicated on ice. After centrifugation with 15000 rpm for 20 minutes at 4° C., the inclusion bodies containing pellet was washed 4 times with 25 ml of buffer (100 mM Tris pH 7.0, 5 mM EDTA, 5 mM DTT, 2% Triton X-100). The pellet was resuspended in 25 ml of buffer (8 M urea, 100 mM Tris pH 8.0, 100 mM DTT) and incubated overnight at room temperature with gently rotating. The resuspended inclusion bodies were dialyzed against 3 l buffer (8 M urea, 100 mM Na₂HPO₄/NaH₂PO₄, 10 mM Tris, 2 mM β-ME, pH8.0) at 4° C. The sample was loaded on Ni²⁺-NTA column (10 ml resuspended agarose; Qiagen) and washed with the same dialysis buffer. Bound protein was eluted using a low pH buffer (8 M urea, 100 mM Na₂HPO₄/NaH₂PO₄, 10 mM Tris, 2 mM 2-mercaptoethanol pH 4.5).

### Example 4

### Refolding of Envelope Protein E Domain III

The eluted protein from Example 3 was dialyzed overnight at 4° C. against 5 l buffer (2 M urea, 50 mM Na₂HPO₄/NaH₂PO₄, 0.5 M arginine, 0.5 mM glutathione (oxidized), 5 mM glutathione (reduced), 10% glycerol, pH 8.5), and dialyzed again overnight at 4° C. in the buffer (50 mM Na₂HPO₄/NaH₂PO₄, 0.5 M arginine, 0.5 mM glutathione (ox), 5 mM glutathione (red), 10% glycerol, pH 8.5). Afterwards, the sample was dialyzed for 4 hours and overnight against buffer (50 mM Na₂HPO₄/NaH₂PO₄, 10% glycerol, pH 8.5).

### Example 5

### Coupling of Envelope Protein E Domain III to Virus-Like Particle of RNA-Bacteriophage Qβ

2.47 g/l virus-like particle of RNA-bacteriophage Qβ (Qβ), wherein said Qβ having amino acid sequence as set forth in SEQ ID NO:1 were derivatized with 2.15 mM SMPH (Pierce, Perbio Science, Lausanne, Switzerland) for 2 hours at 23° C. and then dialyzed against PBS. Then, 0.045 mM envelope protein E domain III from Example 4 and 0.03 mM TCEP (Pierce, Perbio Science, Lausanne, Switzerland) and 0.03 mM derivatized Qβ virus-like particles were incubated for three hours at room temperature. The coupling products were analyzed by SDS-page.

### Example 6

### Immunization

In Experiment 1 C57BL/6 mice were primed with 50 μg Qβ-DIII (serotype 2; from Example 5) on day 0, (subcutaneously; in 0.1 ml PBS) and compared to mice primed with 50 μg Qβ only. In Experiment 2, the vaccine was further purified on a size exclusion column, and then injected together with 1 mg alum, similarly as in Experiment 1. After boosting (Experiment 1: days 14, 28, 87; Experiment 2: days 14, 28, 50) with the same vaccines, respectively, the anti-Qβ and the anti-DIII antibody titers in serum were checked by ELISA at day 169 (Experiment 1) and day 92 (Experiment 2). The IgG titers against the envelope protein E domain III were on average 12952 (Experiment 1) and 29481 (Experiment 2). The Qβ titers were found to be 6652 (Experiment 1) and 125177 (Experiment 2) on average.

### Example 7

### Plaque Reduction Neutralization Test (PRNT50)

The PRNT50 assay was performed according to Russell et al. 1967 (Journal of Immunology 99, 291-296). Plaque count was determined by using LLC-MK₂ plaque assay single overlay technique. Briefly, serum was thawed, diluted, and heat-inactivated by incubation at 56° C. for 30 minutes. Serial 4-fold dilutions of serum were made. An equal volume of diluted virus (40-60 plaque forming units/0.2 ml of Dengue serotype 2 strains; DEN-2 16681, Russell et al 1967, Jpn. J. Med. Sci. Biol. 20 Suppl: 103-108; or DEN-2 S16803 WHO strain, unpublished) was added to each serum dilution tube. Following incubation at 37° C. for 60 minutes, 0.2 ml were removed from each tube and were inoculated onto triplicate 6 well plates of confluent LLC-MK₂ cells. Each plate was incubated at 37° C. for 90 minutes and the monolayer was then overlaid with 4 ml of 3.0% carboxy methyl cellulose/MEM. Plates were incubated for 7 days at 37° C. with 5% CO₂ followed by plaque count. PRNT50 titer was determined by statistical analysis using the SPSS computer program. Eleven sera from mice immunized with Qβ-DIII vaccine (Experiment 1 and Experiment 2) showed PRNT50 titers that were between 67 and 5475 against both isolates. Of the VLP immunized negative control animals (n=8), no sera were found to neutralize; the titers were all below 12 except one serum which had a titer of 65 when challenged with S16803.

### Example 8

### Efficacy of Qβ-DIII in vivo

The Efficacy of Qβ-DIII against dengue virus infection is tested in an animal model as described by Chen et al. 2007 (J. Virol. 81(11):5518-26). C57BL/6 mice are immunized with 50 μg of Qβ-DIII, a dengue serotype 2 vaccine (from Example 5; purified on a size exclusion column) on days 0, 14, 28 (subcutaneously; in 0.2 ml PBS) and compared to mice immunized with 50 μg Qβ only. On day 35, mice are challenged intradermally at four sites on the upper back with PBS or dengue serotype 2 strain 16681 (8×10⁷ plaque forming units (PFU); Russell, Udomsakdi, Halstead, 1967). RNA is extracted from tissues and serum collected three days after viral challenge. The viral capsid gene is amplified by RT-PCR (as described by Chen et al., 2007).

### Example 9

### Tetravalent Mouse Serum Neutralizes all Dengue Serotypes

The DIII domains from serotypes (1 to 4) DEN-1, DEN-2, DEN-3, and DEN-4 were expressed in E. coli and purified to homogeneity. A growth medium containing 1 g/1 methionine was used. After Ni-NTA purification and the subsequent refolding process, proteins were further purified by size exclusion chromatography. In four independent reactions, the coupling of these DIII domains to the RNA-bacteriophage Qβ particle was done similarly as initially for the DV2-DIII domain, these recombinant antigens were rendered highly ordered and repetitive by covalent attachment to a virus-like particle. Uncoupled antigen was removed by size exclusion chromatography and coupling of antigen analyzed by western blot. The tetravalent immunogenic composition was obtained by mixing 50 μg Qβ-DEN-1-EDIII, 50 μg Qβ-DEN-2-EDIII, 50 μg Qβ-DEN-3-EDIII, and 50 μg Qβ-DEN-4-EDIII. C57B1/6 mice were immunized on days 0, 10, and 21 with this tetravalent composition or with these monovalent, unmixed compositions. Alum was used as an adjuvant. On day 28, mice were bled and sera analyzed in a PRNT neutralization assay.

Titers in Table: 1 showed that monovalent and tetravalent vaccine compositions are capable of inducing very high antibody titers in ELISA.

TABLE 1

| | vaccine | EDIII coated | Average ELISA titers |
|---|---|---|---|
| monovalent | Qβ-DEN-1 | DEN-1 | 201864 |
| | Qβ-DEN-2 | DEN-2 | 330661 |
| | Qβ-DEN-3 | DEN-3 | 625853 |
| | Qβ-DEN-4 | DEN-4 | 82258 |
| tetravalent | Qβ-DEN-1234 | DEN-1 | 312659 |
| | Qβ-DEN-1234 | DEN-2 | 498018 |
| | Qβ-DEN-1234 | DEN-3 | 479350 |
| | Qβ-DEN-1234 | DEN-4 | 325569 |

PRNT assay-Neutralization titers: The assay showed sera collected from mice immunized with monovalent or tetravalent compositions neutralized all serotypes in vitro with high average PRNT titers (Table: 2). Qβ negative control sera did not neutralize any dengue serotype and reached titers that were below 10 for all serotypes.

TABLE 2

| | | DEN-1 | DEN-2 | DEN-3 | DEN-4 |
|---|---|---|---|---|---|
| | | (16007) | (16681) | (16562) | (1036) |
| monovalent | Qβ-DEN-1 | 2003 | | | |
| | Qβ-DEN-2 | | 3833 | | |
| | Qβ-DEN-3 | | | 2261 | |
| | Qβ-DEN-4 | | | | 102 |
| tetravalent | Qβ-DEN-1234 | 5604 | 3081 | 938 | 21 |
| control | Qβ | <10 | <10 | <10 | <10 |

### Example 10

### Alternating Ratios of Vaccine Components Lead to Equivalent Immune Response

Alternatively, the four DEN-EDIII proteins from Example 1 were cloned in the same vector but without His₆-tag, and with the following, new C-terminal sequence: —KGSSIGKMGGSCG for serotypes 1, 3 and 4, and following, new C-terminal sequence: —KGSSIGQMGGSCG for serotype-2.

Similarly as described in Example 2, competent E. coli BL21 (DE3) were transformed and a single colony of a kanamycin containing agar plate was grown overnight in liquid culture and used to inoculate 0.3 l of kanamycin containing LB medium. The culture was grown at 37° C. with 150 rpm to OD600 nm=1.0. Expression was induced with 1 mM IPTG. Bacteria were harvested after an overnight induction at 37° C. by centrifuging for 15 minutes at 6000 rpm at 4° C.

The cell pellets were resuspended in 10 ml 20 mM Tris, 2 mM MgCl2, pH8.0 and sonicated on ice. 0.5 mg/ml lysozyme and 20 U/ml benzonase were added to the solution and incubated for 30 minutes at room temperature. After a further sonication, the inclusion bodies were washed in 20 mM Tris, 50 mM NaCl, 4% Triton-X100, 20 mM 2-mercaptoethanol, pH 7.5. The inclusion bodies were resuspended in 8M urea, 50 mM Tris, 20 mM beta-mercaptoethanol, pH 8.5.

The inclusion bodies were refolded by dialyzing two times in the buffer 20 mM Tris, 4.5 mM glutathione (reduced), 0.5 mM glutathione (oxidized), 50 mM NaCl, pH 9.0. After a further dialysis in the same buffer without glutathione, the dialysis was repeated two times in 20 mM Tris, 50 mM NaCl, pH 8.0. The refolded proteins were purified on a gel filtration column (HiLoad 26/60, Superdex 75 pg, GE Healthcare).

AP205 (2 g/l) was derivatized with 1.4 mM SMPH for 30 minutes at room temperature and then dialysed against 20 mM Tris pH8. An equimolar amount of TCEP (Pierce) and envelope protein E domain III (Example 10) were reacted for 20 minutes. Then, 0.057 mM reduced EDIII protein and 0.048 mM derivatized AP205 virus-like particles were incubated for 17 hours at 4° C. The coupling products were analyzed by SDS-page and western blotting with diluted (1:5000) Qβ-DEN-EDIII mouse serum from Example 6.

Groups of 5 mice were immunized three times (on days 0, 11, 20) in the presence of alum with equal amounts of VLP-DEN-1, VLP-DEN-2 and VLP-DEN-3 and an excess of VLP-DEN-4 (20-fold excess of VLP-DEN-4: 10 μg AP205-DEN-1, 10 μg AP205-DEN-2, 10 μg AP205-DEN-3, 200 μg AP205-DEN-4; or 5-fold excess of VLP-DEN-4: 20 μg AP205-DEN-1, 20 μg AP205-DEN-2-EDIII, 20 μg AP205-DEN-3, 100 μg AP205-DEN-4). When immunizing with a 20-fold excess of DEN-4 over DEN-1, DEN-2, DEN-3 vaccines, the IgG ELISA-titers against the EDIII protein were 228,000 for DEN-1, 146,000 for DEN-2, and 108,000 for DEN-3 and 675,000 for DEN-4. When immunizing with a 5-fold excess of DEN-4 over DEN-1, DEN-2, DEN-3 vaccines, the IgG ELISA-titers against the EDIII protein were 331′000 for DEN-1, 306,000 for DEN-2, and 116,000 for DEN-3 and 499,000 for DEN-4.

### Example 11

### Neutralization Assay with Tetravalent Serum from Mice Immunized with Excess of DEN-4 Vaccine

A neutralization experiment is performed as described in example 7. The PRNT-50 neutralization titers against DEN-1, DEN-2, DEN-3, and DEN-4 are determined with sera from example 10.

### Example 12

### Kinetics and Immunogenicity of the Tetravalent AP205-Den-EDIII Vaccine

The four DEN-EDIII proteins with His₆-tag from Example 1 were expressed in 4 l of medium (dYT, 0.05 g/l kanamycin, 0.1% glucose, 1 g/l methionine) as described in example 2. Bacteria were harvested after overnight induction at 37° C. by centrifuging for 15 minutes at 5000 rpm at 4° C.

The cell pellets were resuspended in 60 ml PBS and sonicated on ice. The inclusion bodies were centrifuged at 47000 g for 15 minutes, and resuspended in 60 ml of 20 mM Tris, 2 mM MgCl₂, 1% Triton-X100, 5 mM DTT, pH 8.5. After a further sonication, the inclusion bodies were incubated for 1 hour at 37° C. with 0.2 g/l lysozyme, 2 U/ml benzonase. The inclusion bodies were centrifuged at 47000 g for 15 minutes, and resuspended in 30 ml of 20 mM Tris, 2% Triton-X100, 5 mM DTT, pH 8.5. After centrifugation, the inclusion bodies were resuspended in 8 M urea, 50 mM Tris, 20 mM beta-mercaptoethanol, pH 8.5. Insoluble debris was removed by centrifugation at 17000 g for 30 minutes.

20 ml Fractogel EMD chelate (Catalog number: 1.10338.0250; Merck) was packed on a XK16 column (GE healthcare) and loaded with 100 mM NiSO4. The column was washed with PBS and then with 8 M urea, 50 mM Tris, 20 mM beta-mercaptoethanol, pH 8.0. 1350 mg of protein sample was loaded on the column and washed with 8 M urea, 50 mM Tris, 20 mM beta-mercaptoethanol, pH 8.0, and eluted with 8 M urea, 50 mM Tris, 20 mM beta-mercaptoethanol, 250 mM imidazole, pH 8.0.

200 mg of eluted protein were dialysed two times at 1 mg/ml in 50 mM Tris, 0.4 M L-Arginine, 10% glycerol, 4.5 mM glutathione (reduced), 0.5 mM glutathione (oxidized), 20 mM beta-mercaptoethanol, pH 8.5. Then, dialysis was repeated two times in 20 mM Tris pH 7.5. Precipitated material was removed by centrifugation at 15000 rpm for 10 minutes in a SS34 Sorvall centrifuge. 200 ml of soluble proteins were concentrated to approximately 30 ml by Tangential Flow Filtration (Catalog number: PXB005A50; Biomax 5 kDa, Pellicon XL, 50 cm2, Millipore; 100 ml/minute). Refolded, monomeric proteins were purified on a gel filtration column (HiLoad 26/60, Superdex 75 pg, GE Healthcare) in the buffer 20 mM Tris, pH 7.5.

2.9 g/l AP205 were derivatized with 2.75 mM SMPH for 30 minutes at room temperature and then dialysed against 20 mM Tris pH 7.5. Then, an equimolar amount of TCEP (Pierce) and envelope protein E domain III from Example 12 were preincubated for 17 hours at 4° C. Then, 0.05 mM EDIII protein and 0.052 mM derivatized AP205 virus-like particles were incubated for 2 hours at room temperature. The coupling products were purified on a gel filtration column (HiLoad 26/60, Superdex 75 pg, GE Healthcare) and analyzed by SDS-page and western blotting with diluted (1:2000) Qβ-DEN-EDIII mouse serum from Example 6.

Groups of 6 female C57B1/6 mice were immunized in the presence of alum subcutaneously three times (on days 0, 14, and 28) with 200 μg of the tetravalent AP205-DEN-EDIII mixture (50 μg of each serotype vaccine). Table: 3 shows that EDIII specific ELISA-titers can be reached after only a single administration of the tetravalent AP205-DEN-EDIII conjugate vaccine and finally reach titers above 24′000 for all immobilized EDIII serotype proteins. EDIII-specific antibody titers are expressed as those serum dilutions which lead to half-maximal OD450 nm in ELISA.

TABLE 3

| | preimmune | day 13 | day 27 | day 45 |
|---|---|---|---|---|
| AP205-DEN-1-EDIII | <50 | 5724 | 29605 | 46005 |
| AP205-DEN-2-EDIII | <50 | 1211 | 31616 | 37090 |
| AP205-DEN-3-EDIII | <50 | 2558 | 26931 | 35577 |
| AP205-DEN-4-EDIII | <50 | 827 | 13116 | 24626 |

A neutralization experiment was performed as described in example 7. The PRNT-50 neutralization titers in table: 4 show that these tetravalent sera (day 45) specifically have neutralized DEN-1, DEN-2, DEN-3, and DEN-4 in vitro.

TABLE 4

| | DEN1 | DEN2 | DEN3 | DEN4 |
|---|---|---|---|---|
| DEN-isolate | (16007) | (16681) | (16562) | (1036) |
| PRNT-50 titer | 19761 | 4685 | 2574 | 25 |
| (preimmune serum | | | | |
| background subtracted) | | | | |

### Example 13

### Immunogenicity of the Coupled or Unconjugated EDIII Proteins

The four DEN-EDIII proteins without His₆-tag from Example 10 were expressed in 21 of medium as described in example 10, but bacteria were harvested after 4 hours induction at 37° C. by centrifuging for 15 minutes at 6000 rpm at 4° C.

The cell pellets were resuspended in 45 ml 20 mM Tris, 2 mM MgCl2, pH 7.5 and sonicated on ice. 0.25 mg/ml lysozyme and 5 U/ml benzonase were added to the solution and incubated for 1 hour at 37° C. After the addition of 150 ml 20 mM Tris, 50 mM NaCl, 4% Triton-X100, 20 mM 2-mercaptoethanol, pH 7.5, and a further sonication, the inclusion bodies were centrifuged and resuspended in 20 mM Tris, 50 mM NaCl, 4% Triton-X100, 20 mM 2-mercaptoethanol, pH 7.5. Then, the inclusion bodies were centrifuged again and resuspended in 8 M urea, 50 mM Tris, 20 mM beta-mercaptoethanol, pH 8.5.

The inclusion bodies were refolded at 1 mg/ml by dialyzing once for 40 hours at 4° C. in the buffer 20 mM Tris, 4.5 mM glutathione (reduced), 0.5 mM glutathione (oxidized), 50 mM NaCl, 400 mM L-arginine, 10% glycerol, pH 9.0. After a further dialysis for 8 hours at 4° C. in the buffer 20 mM Tris, 4.5 mM glutathione (reduced), 0.5 mM glutathione (oxidized), 50 mM NaCl, pH 8.0, the dialysis was repeated for 15 hours at 4° C. in 20 mM Tris, 50 mM NaCl, pH 8.0. The refolded, monomeric proteins were purified on a gel filtration column (HiLoad 26/60, Superdex 75 pg, GE Healthcare) in the buffer 20 mM Tris, pH 8.0. Eluted fractions were concentrated on centrifugal filter units (10 kD MWCO, Amicon Ultra-15, Millipore).

2 g/l AP205 were derivatized with 1.4 mM SMPH for 30 minutes at room temperature and then dialysed against 20 mM Tris pH8. Then, an equimolar amount of TCEP (Pierce) and envelope protein E domain III from Example 13 were preincubated for 2 hours at 4° C. Then, 0.029 mM EDIII protein and 0.036 mM derivatized AP205 virus-like particles were incubated for 2 hours at room temperature. The coupling products were purified on a gel filtration column (HiLoad 26/60, Superdex 75 pg, GE Healthcare) and analyzed by SDS-page and western blotting with diluted (1:2000) QβDEN-EDIII mouse serum from Example 6.

Groups of 5 female C57B1/6 mice were immunized subcutaneously three times (with alum; on days 0, 11, and 21) with 200 μg of the tetravalent AP205-DEN-EDIII vaccine (50 μg of each serotype) or with 200 μg of tetravalent, unconjugated mixture of the AP205/EDIII each time. Alternatively, 50 μg of the coupled, monovalent AP205-DEN-EDIII vaccine or a monovalent, unconjugated mixture of the AP205/EDIII was injected each time.

Table: 5 shows that ELISA-titers can be boosted after 28 days up to titers of 560,000-1,200,000. Three injections of the unconjugated, tetravalent AP205/EDIII mixture did result in three to seven fold lower titers than the coupled AP205-DEN-EDIII. Furthermore, three injections of an unconjugated, monovalent AP205/EDIII mixture did result in 10 to 38 fold lower titers than the coupled monovalent AP205-DEN-EDIII vaccines (Table: 4). DEN-EDIII-specific antibody titers are expressed as those serum dilutions which lead to half-maximal OD450ₙₘ in ELISA. Shown are mean titers.

TABLE 5

| | vaccine | Coated antigen | coupled | mixed |
|---|---|---|---|---|
| MonoValent | AP205-DEN-1-EDIII | DEN-1-EDIII | 504468 | 13149 |
| | AP205-DEN-2-EDIII | DEN-2-EDIII | 272339 | 22911 |
| | AP205-DEN-3-EDIII | DEN-3-EDIII | 190046 | 19670 |
| | AP205-DEN-4-EDIII | DEN-4-EDIII | 126558 | 4145 |
| TetraValent | AP205-DEN-1234-EDIII | DEN-1-EDIII | 1026480 | 197075 |
| | AP205-DEN-1234-EDIII | DEN-2-EDIII | 630357 | 84053 |
| | AP205-DEN-1234-EDIII | DEN-3-EDIII | 1156246 | 414087 |
| | AP205-DEN-1234-EDIII | DEN-4-EDIII | 560357 | 101584 |

### Example 14

### Tetravalent Vaccine in a Primate Dengue Model

The efficacy of the tetravalent AP205-DENV-EDIII vaccine against dengue virus infection is tested in a primate model as described by Blaney et al., 2008 (Vaccine, vol. 26, page 817). A group of 4 rhesus macaques is immunized with 600 μg of the tetravalent AP205-DENV-EDIII (150 μg of each serotype; from Example 13) on days 0, 14, 28 (subcutaneously, with alum) and compared to monkeys immunized with 600 μg AP205 only. On day 35, all animals are challenged by subcutaneous infection with 105 PFU of DENV-3 Sleman/78 wild type virus. Serum is collected on days 0-6, 8, and 10, frozen at −80° C., and the virus titer in serum samples is determined by plaque assay in Vero cells.

Serum aliquots are thawed and serial 10-fold dilutions are inoculated onto Vero cell monolayer cultures in 24-well plates (Durbin et al., 2001, Am. J. Trop. Med. Hyg., vol. 65, page 405). After a 1-hr incubation at room temperature, the monolayers are overlaid with 0.8% methylcellulose in OptiMEM (Life Technologies) supplemented with 5% FBS. Following incubation at 37° C. for four days, virus plaques are visualized by immunoperoxidase staining Briefly, cell monolayers are fixed in 80% methanol for 30 min and rinsed with antibody buffer (5% nonfat milk in PBS). Polyclonal, rabbit DENV-3 specific antibodies (Abcam) are diluted in antibody buffer and added to each well followed by a 1-hr incubation at 37° C. Primary antibody is removed and the cell monolayers are washed twice with antibody buffer. Peroxidase-labeled goat-anti-rabbit IgG (Kirkegaard and Perry Laboratories, Gaithersburg, Md.) is diluted 1:500 in antibody buffer and added to each well, followed by a 1-hr incubation at 37° C. Secondary antibody is removed and the wells are washed twice with PBS. Peroxidase substrate (4-chloro-1-naphthol in H2O2) is added to each well and visible plaques are counted.

### Example 15

### Tetravalent Compositions in a Neutralization Assay

Four new DEN-4-EDIII (serotype-4) proteins from two different DEN-4 strains (strain MY01-23314; Swissprot: Q8B0G5; and NCBI-accession number: U18429, strain: Indonesia 1976, Isolate: 1036) are cloned in the same vector as in example 1, but without His 6-tag, and with the following, new N-terminal sequences MSYTMCS-(MY01-23314) and MSYTMCP-(Indonesia 1976). The new C-terminal sequences are -WFRKGSSIGKGSCG (MY01-23314), and -WFRKGSSGSCG (MY01-23314) and -WFRKGSSIGKGSCG (Indonesia 1976) and -WFRKGSSGSCG (Indonesia 1976).

These new four new proteins are then expressed and purified as described in example 13. Tetravalent vaccine compositions are prepared with equal amounts of each serotype vaccine (50 μg of each serotype AP205-DEN-EDIII) or with AP205-DEN-4-EDIII vaccine in excess, similarly as described in example 10. Sera from immunized mice are tested by ELISA, similarly as described in example 13. A neutralization experiment is performed as described in example 7.

1. A composition comprising:
(a) a virus-like particle with at least one first attachment site, wherein said virus-like particle is a virus-like particle of an RNA bacteriophage; and
(b) at least one antigen with at least one second attachment site, wherein said at least one antigen is a dengue antigen, wherein said dengue antigen comprises at least position 9 to 99 of domain III of the dengue virus envelope protein E;
and wherein (a) and (b) are linked through said at least one first and said at least one second attachment site.

2.-5. (canceled)

6. The composition of claim 1, wherein said dengue antigen comprises position 9 to 112 of domain III of the dengue virus envelope protein E.

7. (canceled)

8. The composition of claim 1, wherein said dengue antigen comprises position 9 to 109 or position 9 to 112 of any one of the SEQ ID NOs 21, 24, 27, 30 or 32.

9. (canceled)

10. The composition of claim 1, wherein said virus-like particle comprises recombinant coat proteins, mutants or fragments thereof, of an RNA bacteriophage.

11. The composition of claim 10, wherein said RNA bacteriophage is selected from the group consisting of:
(a) bacteriophage Qβ;
(b) bacteriophage AP205;
(c) bacteriophage fr; and
(d) bacteriophage GA.

12. The composition of claim 1, wherein said first attachment site is linked to said second attachment site via at least one covalent bond.

13. The composition of claim 12, wherein said at least one covalent bond is a non-peptide bond.

14.-16. (canceled)

17. The composition of claim 1, wherein said first attachment is an amino group of a lysine, and wherein said second attachment site is a sulfhydryl group of a cysteine.

18.-19. (canceled)

20. The composition of claim 1, wherein only one of said second attachment sites associates with said first attachment site through at least one non-peptide covalent bond leading to a single and uniform type of binding of said antigen to said virus-like particle, wherein said only one second attachment site that associates with said first attachment site is a sulfhydryl group, and wherein said antigen and said virus-like particle interact through said association to form an ordered and repetitive antigen array.

21. The composition of claim 1, wherein said virus-like particle comprises recombinant coat proteins, mutants or fragments thereof, of an RNA bacteriophage, and wherein said at least one antigen is fused to the N- or the C-terminus of said recombinant coat proteins, mutants or fragments thereof.

22. (canceled)

23. The composition of claim 21, wherein said RNA bacteriophage is selected from the group consisting of
(a) bacteriophage AP205;
(b) bacteriophage fr; and
(c) bacteriophage GA.

24. The composition of claim 1, wherein said at least one antigen with said at least one second attachment site further comprises a linker, wherein said linker comprises said second attachment site, and wherein said linker is associated to said antigen by way of one peptide bond.

25. The composition of claim 1, wherein at least one antigen with said at least one second attachment site comprises any one of SEQ ID NOs 22, 25, 28 and 31.

26. A vaccine composition comprising the composition of claim 1.

27. (canceled)

28. A pharmaceutical composition comprising:
(a) the composition of claim 1; and
(b) a pharmaceutically acceptable carrier.

29. (canceled)

30. A method of treating, ameliorating, or preventing dengue fever, dengue hemorrhagic fever, and/or dengue shock syndrome, said method comprising administering an immunologically effective amount of the composition of claim 1 to an animal.

31.-32. (canceled)

33. The composition of claim 1, wherein said domain III of the dengue virus envelope protein E is selected from the group consisting of: (i) domain III of the dengue virus envelope protein E of a dengue virus of serotype-1; (ii) domain III of the dengue virus envelope protein E of a dengue virus of serotype-2; (iii) domain III of the dengue virus envelope protein E of a dengue virus of serotype-3; and (iv) domain III of the dengue virus envelope protein E of a dengue virus of serotype-4.

34. The composition of claim 1, wherein said virus-like particle comprises recombinant coat proteins of RNA bacteriophage Qβ.

35. The composition of claim 34, wherein said coat proteins consist of the amino acid sequence of SEQ ID NO:1.

36. The composition of claim 1, wherein said virus-like particle is a virus-like particle of RNA bacteriophage Qβ.
